(19) United States
(12) Patent Application Publication
(10) Pub. No.: US 2007/0054884 A1
(43) Pub. Date: Mar. 8, 2007

(54) 4-SUBSTITUTED 2-ARYLOXYPHENOL DERIVATIVES AS ANTIBACTERIAL AGENTS

(75) Inventors: Liren Huang, Gaithersburg, MD (US); Joanna Clancy, Gaithersburg, MD (US); Alenka Tomazic, Gaithersburg, MD (US); Weitong Wang, Gaithersburg, MD (US); Christopher Taylor, Gaithersburg, MD (US); W. James Jackson, Gaithersburg, MD (US); Yufa Liu, Gaithersburg, MD (US)

Correspondence Address: FOLEY AND LARDNER LLP;SUITE 500, 3000 K STREET NW, WASHINGTON, DC, 20007, US

(73) Assignee: Emergent Product Development Gaithersburg Inc.

(21) Appl. No.: 11/469,291

(22) Filed: Aug. 31, 2006

Related U.S. Application Data
(60) Provisional application No. 60/813,618, filed on Aug. 31, 2005.

(51) Int. Cl. A61K 31/69; A61K 31/541; A61K 31/5377; A61K 31/496; A61K 31/454; A61K 31/433; A61K 31/427; A61K 31/4245; A61K 31/4196; A61K 31/4192; A61K 31/4178

(52) U.S. Cl. 514/64; 514/210.2; 514/254.02; 514/254.05; 514/326; 514/235.2; 514/227.5; 514/278; 514/252.05; 514/365; 514/364; 514/362; 514/363; 514/359; 514/381; 514/397; 544/60; 544/132; 544/134; 544/138; 546/209; 546/207; 548/110; 548/128; 548/131; 548/253; 548/146; 546/215; 546/255; 548/311.1

(57) ABSTRACT

Antimicrobial compounds, compositions and methods of treatment administering same, of 2-aryloxyphenol derivatives having heterocyclic groups or highly polar functional groups substituted at position 4 of the phenolic ring, as well as methods for their preparation and formation, wherein the compounds are generally of Formula 1.

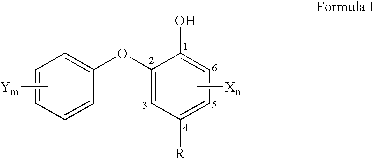

### FIELD OF THE INVENTION

This invention relates to novel substituted 2-aryloxyphenol derivatives possessing a heterocyclic or polar functional substitution attached through a N—C or C—C bond at the para position of hydroxyl group on phenyl ring. More particularly a 5-membered heterocyclic aromatic ring has 1-4 atoms of nitrogen, oxygen and sulfur. The compounds are useful antimicrobial agents, effective against a number of human and bioterrorism pathogens, including staphylococci, streptococci and enterococci as well as Bacillus anthracis and Bacillus cereus.

### BACKGROUND OF THE INVENTION

Drug resistance of existing antimicrobial and particularly antibacterial agents is a clinical problem worldwide. A number of approaches have been taken by the pharmaceutical community to combat the alarming bacterial resistance problem. One approach is the structural modification of known antibiotics to overcome resistance liabilities. A second approach is combination therapies, for example, the combination of antibiotics with drugs that inhibit the enzyme or protein that causes a particular resistance. Although these approaches have met with some success, the best solution to the bacterial resistance dilemma remains the identification of novel antibacterial agents employing a unique mechanism of action.

The chemical and biological literature abounds with reports about 2-aryloxyphenol (A) due in great part to their antibacterial activities. Many of the compounds were initially used in the treatment of textiles, and there have been hundreds of patents filed worldwide for their incorporation into a diverse range of products over the last 30 years. Triclosan (B) is the most potent and widely used member of this class of antibacterial and antifungal agents, and is used in products such as antiseptic soaps, toothpastes, fabrics and plastics.

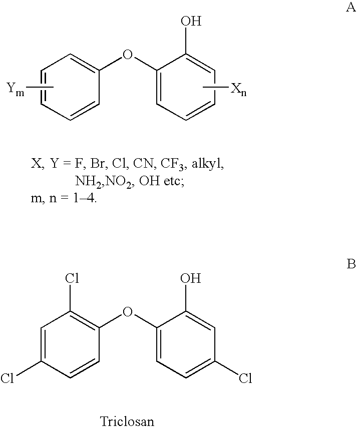

The patents U.S. Pat. No. 3,506,720, U.S. Pat. No. 3,903,007, NR 432119, DE 2800105 A1 and CH 460443 describe the usefulness of halogenated 2-aryloxyphenols, especially 5-chloro-(2,4-dichloro-phenoxy)-phenol (triclosan), and corresponding esters in antibacterial compositions and methods for the protection of organic materials, films and textile fibers.

The patent U.S. Pat. No. 5,185,377, U.S. Pat. No. 6,204,230, U.S. Pat. No. 6,107,261, U.S. Pat. No. 6,136,771 and WO 98/55096 describe the pharmaceutical compositions which comprise triclosan and other 2-aryloxyphenols useful in treatment of bacterial infections, inflammatory disease, and spasmolytic disease.

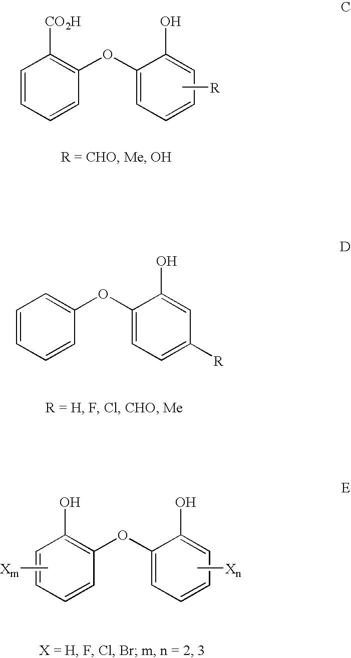

Syntheses of compounds of type C, where one of the benzene rings is substituted with a 2-carboxylic group, have been reported by Fujikawa (Yakugaku Zasshi (1963), 63, 1172) but there is no biological activity data reported. Health and Sivaraman have reported the relation of the antibacterial activities of triclosan and its related compounds, D, to inhibition of the bacterial enoyl reductase Fab I. (J. Bio. Chem., 1998, 273:3016; J. Med. Chem., 2004, 47:509). Studies on the synthesis, activity and molecular modeling of type E compounds as human aldose reductase inhibitors have been described. (J. Med. Chem., 2003, 46:5208)

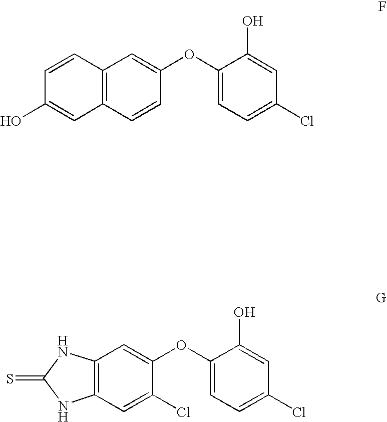

Antimalarial activities of triclosan and compound F associated with their inhibitory activity against malarial enoyl carrier protein reductase have been described by Perozzo, R. (J. Bio. Chem., 2002, 277:13106). The patent U.S. Pat. No. 4,205,077 described cyclic thiourea derivatives of 2-aryloxyphenol including compound G useful as anthelmintic agents in animals.

Triclosan had long been thought of as a nonspecific biocide that disrupts cell membranes, rendering bacteria unable to assimilate nutrients and to proliferate. This view has been changed recently by McMurry, Health et al who discovered that triclosan and other members of the 2-aryloxyphenols, such as compound D, directly target Fab I, the enoyl-acyl carrier protein reductase of type II bacterial fatty acid synthesis. (Nature 1998, 394:531; J. Bio. Chem. 1998, 273:3016; J. Med. Chem. 2004, 47:509). This was followed by analyses of the crystal structure of the Escherichia coli Fab I—NAD+—triclosan complex and computational chemistry thereafter (Biochemistry, 2003, 42:4406; J. Bio. Chem., 2002, 277:13106; Protein Sci., 1999, 8:2529) laid the foundation for rational drug design in the area of 2-aryloxyphenol antibacterial agents.

The present invention includes the design and synthesis of 4-substituted-2-aryloxyphenol derivatives by incorporation of heterocyclic or highly polar functional groups in order to improve their water solubility, bio-availability and microbial activity in vivo.

### DETAILED DESCRIPTION OF THE INVENTION

The present invention includes compounds and compositions of Formula I or a pharmaceutically acceptable salt thereof, 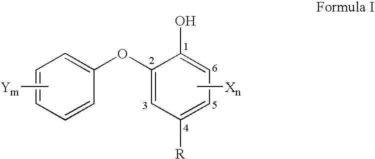
wherein
X and Y are each independently chosen from halogen, CN, OH, NH₂, NMe₂, NO₂, SO₂Me, SO₃H, SO₂NH₂, CHO, CO₂NH₂, CH(NOMe), C(O)Me, CO₂Me, CO₂Et, C₁-C₄ alkyl and cycloalkyl, CF₃, SMe, OMe, OEt etc.; m is 0, 1, 2, 3, 4 or 5, and n is 0, 1, 2 or 3,
R is chosen from
1) —B(OH)₂,
2) —CO₂H,
3) —CONH₂,
4) —C(NH)NH₂,
5) —C(NOH)NH₂,
6) —C(NNH₂)NH₂,
7) —C(O)NHOH,
8) —CONHNH₂,
9) —NHNH₂,
10) —NHC(NH)NH₂,
11) —R¹,
12) —NHC(O)R¹,
13) —NHSO₂R¹,
14) —NHSO₂R²,
15) —NHC(O)NHR²,
16) —NHC(S)NHR²,
17) [2-(2-methyl-5-nitro-imidazol-1-yl)-ethyl]oxycarbonylamino or [2-(2-methyl-5-nitro-imidazol-1-yl)-ethyl]oxycarbonyl,
18) —R³,
   wherein
   R¹ is a heterocycle of from 5 to 8 atoms with 1-4 heteroatoms chosen from nitrogen, oxygen, or sulfur or phenyl, all of which rings may be optionally substituted up to 3 times by halogen, OH, NH₂, NO₂, NMe₂, NHAc, Me, Et, SMe, OMe, OEt, CHO, CN, CH₂OH, CO₂H, CONH₂, CO₂Me, CO₂Et, SO₂Me etc.
   R² is H, C₁-C₄ alkyl, C₃-C₇ cycloalkyl, —CO₂Me, —CO₂Et, 2-oxo-tetrahydro-furan-3-yl, 3-pyridinylcarbonyl; phenyl group substituted up to two times with halogen, CN, OH, OMe, SMe, Me, Et, cyclopropyl, CF₃, NH₂, NMe₂, NO₂, CO₂Et, CO₂Me, CO₂H, SO₂Me, SO₂NH₂ etc. or R³ on the ring.
   R³ is independently, but not limited to, azetidin-1-yl, 3-amino-azetidin-1-yl, pyrrolidin-1-yl, 3-amino-pyrrolidin-1-yl, 3-amino-4-methyl-pyrrolidin-1-yl, 7-amino-5-aza-spiro[2.4]hept-5-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl and thiomorpholin-4-yl etc.

More preferred compounds of the present invention are those of above Formula I 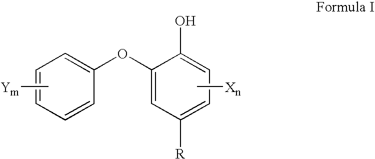
wherein
X and Y are each independently chosen from halogen, CN, OH, NH₂, NMe₂, NO₂, SO₂Me, SO₃H, SO₂NH₂, CHO, CO₂NH₂, CH(NOMe), C(O)Me, CO₂Me, CO₂Et, C₁-C₄ alkyl and cycloalkyl, CF₃, SMe, OMe, OEt etc.; m is 0, 1, 2, 3, 4 or 5, and n is 0, 1, 2 or 3.
R is chosen from
1) —B(OH)₂,
2) —CO₂H,
3) —CONH₂,
4) —C(NH)NH₂,
5) —C(NOH)NH₂,
6) —C(NNH₂)NH₂,
7) —C(O)NHOH,
8) —CONHNH₂,
9) —NHNH₂,
10) —NHC(NH)NH₂,
11) —R¹,
12) —NHC(O)R¹,
13) —NHSO₂R¹,
14) —NHSO₂R²,
15) —NHC(O)NHR²,
16) —NHC(S)NHR²,
17) [2-(2-methyl-5-nitro-imidazol-1-yl)-ethyl]oxycarbonylamino or [2-(2-methyl-5-nitro-imidazol-1-yl)-ethyl]oxycarbonyl,
18) —R³.
   wherein

R¹ is
(a) Substituted furanyl: 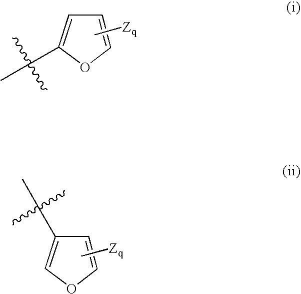
(b) Substituted thiophenyl: 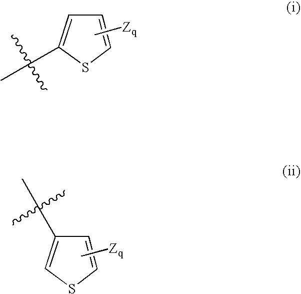
(c) Substituted pyrrolyl: 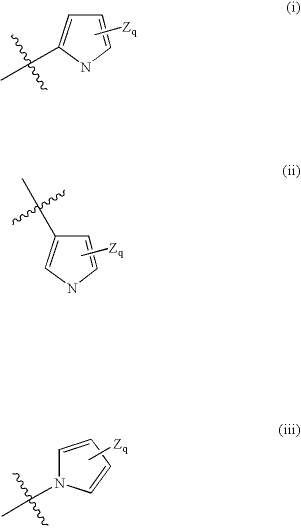
(d) Substituted isoxazolyl 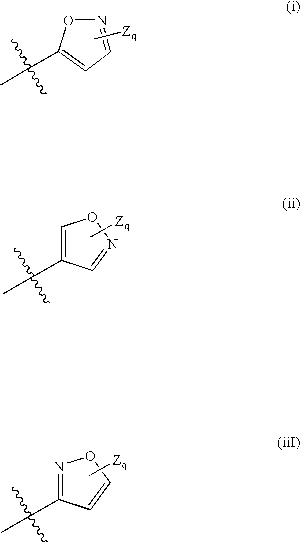
(e) Substituted isothiazolyl 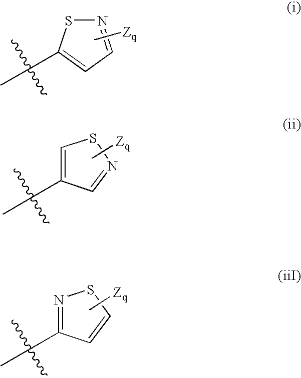
(f) Substituted pyrazolyl 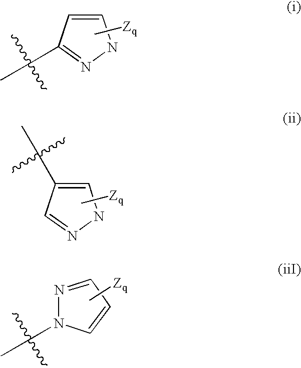
(g) Substituted oxazolyl 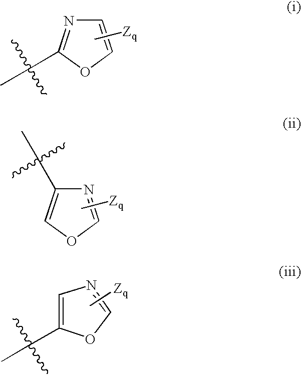
(h) Substituted thiazolyl 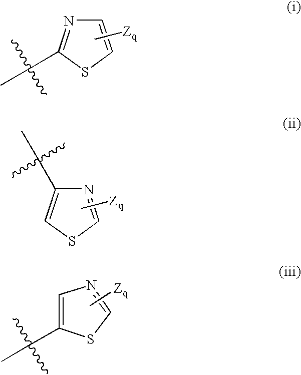
(i) Substituted imidazolyl 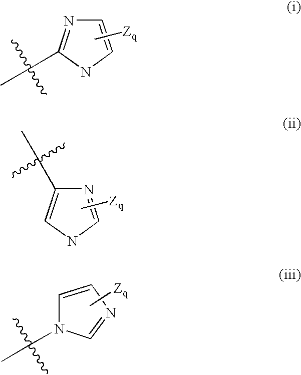
(j) Substituted 1H-[1,2,3]triazolyl 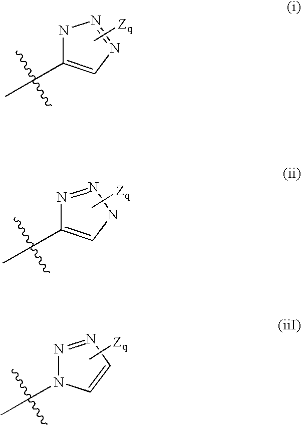
(k) Substituted 2H-[1,2,3]triazol-2-yl 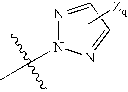
(l) Substituted [1,2,3]oxadiazolyl 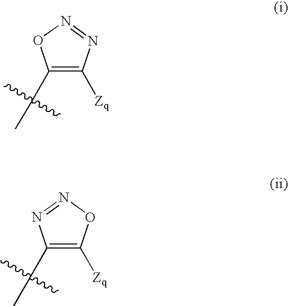
(m) Substituted [1,2,3]thiadiazolyl 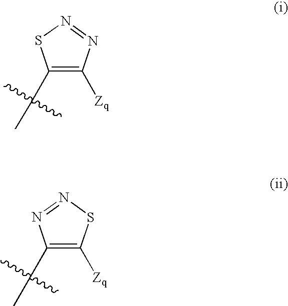
(n) Substituted 4H-[1,2,4]triazolyl 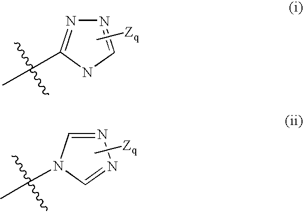
(o) Substituted 1H-[1,2,4]triazolyl 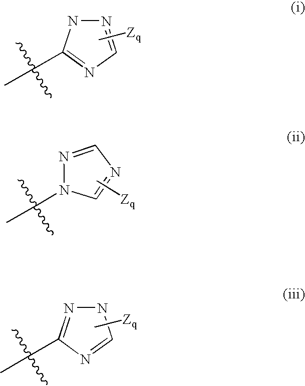
(p) Substituted [1,3,4]oxadiazolyl 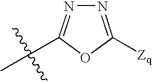
(q) Substituted [1,3,4]thiadiazolyl 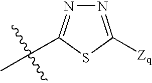
(r) Substituted [1,2,4]oxadiazolyl 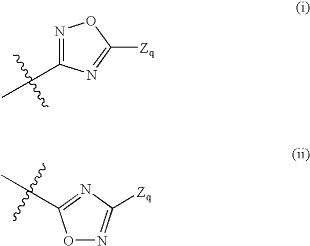
(s) 1H-tetrazol-5-yl (i) or 2H-tetrazol-5-yl (ii) 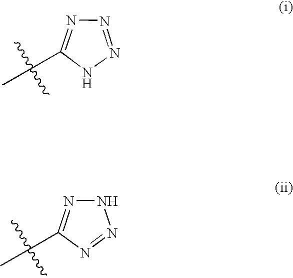
(t) 1H-tetrazol-1-yl 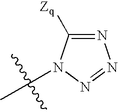
(u) 5-oxo-4H-[1,2,4]oxadiazol-3-yl 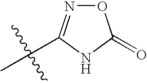
(v) Substituted 4,5-dihydro-thiazol-2-yl and 5,6-dihydro-4H-[1,3]thiazin-2-yl 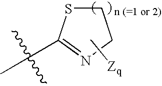
(w) Substituted pyridazinyl

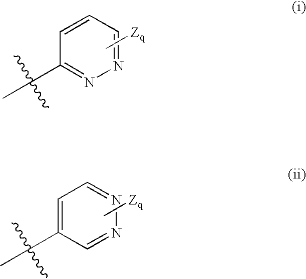

Here, Z is independently chosen from F, Cl, OH, NH₂, NO₂, NMe₂, NHAc, Me, Et, SMe, OMe, OEt, CHO, CN, CH₂OH, CO₂H, CONH₂, CO₂Me, CO₂Et, SO₂Me etc.; q is 0, 1, 2 or 3.

R² is H, C₁-C₄ alkyl, C₃-C₇ cycloalkyl, —CO₂Me, —CO₂Et, 2-oxo-tetrahydro-furan-3-yl, 3-pyridinylcarbonyl; phenyl group substituted up to two times with F, Cl, Br, CN, OH, OMe, SMe, Me, Et, cyclopropyl, CF₃, NH₂, NMe₂, NO₂, CO₂Et, CO₂Me, CO₂H, SO₂Me, SO₂NH₂ etc. or R³ on the ring.

R³ is independently but not limited to azetidin-1-yl, 3-amino-azetidin-1-yl, pyrrolidin-1-yl, 3-amino-pyrrolidin-1-yl, 3-amino-4-methyl-pyrrolidin-1-yl, 7-amino-5-aza-spiro[2.4]hept-5-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl and thiomorpholin-4-yl etc.

More preferred, the present invention provides compounds of Formula I,

wherein

X and Y are each independently F, Cl, CN, OH, NH₂, NO₂, SO₂NH₂, CO₂NH₂, CH(NOMe), C(O)Me, CO₂Me, CO₂Et, methyl, ethyl, cyclopropyl, CF₃, SMe, OMe, OEt etc.; m is 0, 1, 2, 3 or 4 and n is 0, 1 or 2.

R is chosen from

1) —B(OH)₂,
2) —CO₂H,
3) —CONH₂,
4) —C(NH)NH₂,
5) —C(NOH)NH₂,
6) —C(NNH₂)NH₂,
7) —CONHNH₂,
8) —NHNH₂,
9) —NHC(NH)NH₂,
10) —R¹,
11) —NHC(O)R¹,
12) —NHSO₂R¹,
13) —NHSO₂R²,
14) —NHC(O)NHR²,
15) —NHC(S)NHR²,
16) —R³,
   wherein,
   R¹ is (a), (b), (c)-(i) and (iii), (d), (f), (g), (h), (i), (j), (m)-(i), (n), (O), (p), (q), (r), (s), (t), (u), (v). Here, Z is independently chosen from F, Cl, OH, NH₂, NHAc, Me, Et, SMe, OMe, OEt, CHO, CN, CH₂OH, CO₂H, CONH₂, CO₂Me, CO₂Et, SO₂Me etc.; q is 0, 1, 2 or 3.
   R² is H, methyl, ethyl, cyclopropyl, methylcyclopropyl, cyclopropylmethyl, cyclobutyl, cyclopentyl, cyclohexyl, —CO₂Me, —CO₂Et, 2-oxo-tetrahydro-furan-3-yl, phenyl group substituted up to two times with F, Cl, Br, CN, OH, OMe, SMe, Me, Et, cyclopropyl, CF₃, NMe₂, NO₂, CO₂Et, CO₂Me, SO₂Me, SO₂NH₂ etc. or R³ on the ring.
   R³ is independently but not limited to azetidin-1-yl, 3-amino-azetidin-1-yl, pyrrolidin-1-yl, 3-amino-pyrrolidin-1-yl, 3-amino-4-methyl-pyrrolidin-1-yl, 7-amino-5-aza-spiro[2.4]hept-5-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl and thiomorpholin-4-yl etc.

The present invention includes pharmaceutical compositions which comprise an antibacterially effective amount of compound for Formula I or a pharmaceutically acceptable salt thereof with pharmaceutical acceptable carriers.

Preferred Compounds

The following compounds are preferred:

1. 5-Chloro-2-(2,4-dichlorophenoxy)-4-morpholin-4-yl-phenol;
2. 5-Chloro-2-(2,4-dichloro-phenoxy)-4-(4-methyl-piperazin-1-yl)-phenol;
3. 5-Chloro-2-(2,4-dichloro-phenoxy)-4-thiophen-2-yl-phenol;
4. 5-Chloro-2-(2,4-dichloro-phenoxy)-4-furan-2-yl-phenol;
5. 5-Chloro-2-(2,4-dichloro-phenoxy)-4-thiophen-3-yl-phenol;
6. 2-Chloro-5-(2,4-dichloro-phenoxy)-4-hydroxy-phenylboronic acid;
7. 1-[2-chloro-5-(2,4-dichlorophenoxy)-4-hydroxyphenyl-3-(ethoxycarbonyl)thiourea;
8. 1-[2-Chloro-5-(2,4-dichloro-phenoxy)-4-hydroxy-phenyl]-3-(furan-2-carbonyl)-thiourea;
9. 1-[4-Hydroxy-3-(2-hydroxy-phenoxy)-phenyl]-3-(ethylozycarbonyl)thiourea;
10. 1-[2-Chloro-5-(2,4-dichloro-phenoxy)-4-hydroxy-phenyl]-3-(4-fluoro-phenyl)-thiourea;
11. 1-[2-Chloro-5-(2,4-dichloro-phenoxy)-4-hydroxy-phenyl]-3-(4-methoxy-phenyl)-thiourea;
12. 1-[2-Chloro-5-(2,4-dichloro-phenoxy)-4-hydroxy-phenyl]-3-cyclohexyl-thiourea;
13. 1-[2-Chloro-5-(2,4-dichloro-phenoxy)-4-hydroxy-phenyl]-3-(4-nitro-phenyl)-thiourea;
14. 1-[2-Chloro-5-(2,4-dichloro-phenoxy)-4-hydroxy-phenyl]-3-(2-oxo-tetrahydro-furan-3-yl)-thiourea;
15. 1-(3,5-Bis-trifluoromethyl-phenyl)-3-[2-chloro-5-(2,4-dichloro-phenoxy)-4-hydroxy-phenyl]-urea;
16. 1-(3,5-Bis-trifluoromethyl-phenyl)-3-[2-chloro-5-(2,4-dichloro-phenoxy)-4-hydroxy-phenyl]-thiourea;
17. 5-Chloro-2-(2,4-dichloro-phenoxy)-4-pyrrol-1-yl-phenol;
18. 2-(2-Hydroxy-5-pyrrol-1-yl-phenoxy)-benzonitrile;
19. Thiophene-2-carboxylic acid [2-chloro-5-(2,4-dichloro-phenoxy)-4-hydroxy-phenyl]-amide;
20. Furan-2-carboxylic acid [2-chloro-5-(2,4-dichloro-phenoxy)-4-hydroxy-phenyl]-amide;
21. 4-Methyl-[1,2,3]thiadiazole-5-carboxylic acid [2-chloro-5-(2,4-dichloro-phenoxy)-4-hydroxy-phenyl]-amide;
22. 5-Methyl-isoxazole-3-carboxylic acid [2-chloro-5-(2,4-dichloro-phenoxy)-4-hydroxy-phenyl]-amide;
23. N-{5-[2-Chloro-5-(2,4-dichloro-phenoxy)-4-hydroxy-phenylsulfamoyl]-4-methyl-thiazol-2-yl}-acetamide;
24. 1H-Imidazole-4-carboxylic acid [2-chloro-5-(2,4-dichloro-phenoxy)-4-hydroxy-phenyl]-amide;
25. 1H-Pyrazole-4-carboxylic acid [2-chloro-5-(2,4-dichloro-phenoxy)-4-hydroxy-phenyl]-amide;
26. 2-(2-Hydroxy-4-methyl-5-thiophen-2-yl-phenoxy)-benzonitrile;
27. 2-(2-Hydroxy-4-methyl-5-thiophen-3-yl-phenoxy)-benzonitrile;
28. 2-(5-Furan-2-yl-2-hydroxy-4-methyl-phenoxy)-benzonitrile;
29. 2-(2-Hydroxy-4-methyl-5-pyrrol-1-yl-phenoxy)-benzonitrile;
30. 5-Chloro-2-(4-fluoro-2-hydroxy-5-morpholin-4-yl-phenoxy)-benzonitrile;
31. 2-(2-Hydroxy-4-methyl-5-morpholin-4-yl-phenoxy)-benzonitrile;
32. 5-Chloro-2-(2,4-dichloro-phenoxy)-4-tetrazol-1-yl-phenol;
33. 5-Chloro-2-(2,4-dichloro-phenoxy)-4-(1H-tetrazol-5-yl)-phenol;
34. 2-Chloro-5-(2,4-dichloro-phenoxy)-4,N-dihydroxy-benzamidine;
35. 3-[2-Chloro-5-(2,4-dichloro-phenoxy)-4-hydroxy-phenyl][1,2,4]oxadiazole-5-carboxylic acid ethyl ester;
36. 3-[2-Chloro-5-(2,4-dichloro-phenoxy)-4-hydroxy-phenyl]-[1,2,4]oxadiazole-5-carboxylic acid;
37. 2-Chloro-5-(2,4-dichloro-phenoxy)-4-hydroxy-benzoic acid;
38. 2-Chloro-5-(2,4-dichloro-phenoxy)-4-hydroxy-benzamide;
39. 4-(5-Amino-[1,3,4]thiadiazol-2-yl)-5-chloro-2-(2,4-dichloro-phenoxy)-phenol.

Descriptions of the compounds of the present invention rely upon terms that include the following.

The compounds of the invention are named according to the IUPAC or CAS nomenclature system. The carbon atom content of various hydrocarbon-containing moieties is indicated by a prefix designating the minimum and maximum number of carbon atoms in the moiety, i.e., the prefix Ci-Cj indicates a moiety of the integer “i” to the integer “j” carbon atoms, inclusive. Thus, for example, C1-C4 alkyl and cycloalkyl refers to alkyls and cycloalkyls of one to four carbon atoms, inclusive, or methyl, ethyl, propyl, isopropyl, cyclopropyl, butyl and its isomeric forms, and cyclobutyl, cyclopropylmethyl and methylcyclopropyl.

Hydroxyl protecting groups (PG) are benzyl, 4-methoxybenzyl, methyl, benzyl, 2,2,2-trichloroethyl, t-butyldimethylsilyl, trimethylsilyl, t-butyl, allyl, or as described in Greene, Theodora W., Protective Groups in Organic Synthesis, 1999, John Wiley & Sons Inc.: Chapter 3.

Unless otherwise specified, the terms “heterocycle”, “heterocyclic group”, or heterocyclic” are used interchangeably herein and includes monocyclic, bicyclic ring or bridged ring system having from 4-10 atoms, 1-4 of which are selected from oxygen, sulfur and nitrogen. Heterocyclic group includes non-aromatic groups such as morpholin-4-yl and 4-methyl-piperazin-1-yl, and heteroaryl groups such as thiophenyl and oxadiazolyl. The term “aryl” in “heteroaryl” refers to aromaticity, a term known to those skilled in the art and defined in greater detail in “Advanced Organic Chemistry”, M. B. Smith and J. March, 5^{th} Ed., John Wiley & Sons, New York, N.Y. (2001). Preferred heterocyclic groups represented by the term are R¹, wherein the waved line indicates the bond of attachment. For example, a bond pointing inside a ring such as −Zq in (f)-(i) indicates that the substituent is able to connect to any carbon and nitrogen on the ring that can accept a covalent bond other than hydrogen. Heterocyclic groups in the compounds of the invention may be C-attached or N-attached where such is possible.

As is apparent to those of ordinary skill in the art, the compounds of the present invention can exist in tautomeric forms, and all such tautomeric forms are included within the scope of the present invention. For instance, in the compounds of Example 33, the 1H-tetrazolyl group can exist as the 2H-tetrazol-5-yl group and both such tautomers are included within the scope of the present invention. Geometric isomers of olefins, C═N double bonds and the like can also be present in the compounds described herein, and all such stable isomers are contemplated in the present invention.

It will be apparent to one skilled in the art that selected heterocyclic ring systems may have chiral centers present to give rise to enantiomers and diastereomers. These diastereomers and enantiomers, in racemic, diastereomerically or enantiomerically enriched forms, are also within the scope of the compounds of the invention.

The compounds of the invention are capable of forming both pharmaceutically acceptable acid addition and/or base salts. Base salts are formed with metals or amines, such as alkali and alkaline earth metals or organic amines. Examples of metals used are sodium, potassium, magnesium, calcium, and the like. Examples of suitable amines are N,N′-dibenzylethyldiamine, chloroprocaine, choline, diethanolamine, ethylenediamine, N-methylglucamine, and procaine.

Pharmaceutically acceptable acid addition salts are formed with organic or inorganic acids. Examples of suitable acids for salt formation are hydrochloric, sulfuric, phosphoric, acetic, citric, oxalic, malonic, salicyclic, malic, gluconic, fumaric, succinic, ascorbic, maleic, methanesulfonic, and the like. The salts are prepared by contacting the free base form with a sufficient amount of the desired acid to produce either mono or di, etc. salt in the conventional manner. The free base forms may be regenerated by treating the salt form with a base. For example, dilute solutions of aqueous base may be utilized. Dilute aqueous sodium hydroxide, potassium carbonate, ammonia, and sodium bicarbonate solutions are suitable for this purpose. The free base forms differ from their respective salts forms somewhat in certain physical properties such as solubility in polar solvents, but the salts are otherwise equivalent to their respective free base forms for purposes of the invention.

The compounds of the invention are capable of forming pharmaceutically acceptable prodrugs. “Prodrugs” are considered to be any covalently bonded carriers which release the active parent drug in vivo when such prodrug is administered to a subject. Prodrugs of a compound are prepared by modifying functional groups present in the compounds in such a way that the bonds are cleaved, either in routine manipulation or in vivo, to the parent compounds. Prodrugs include, but are not limited to, compounds wherein hydroxyl, amine, or sulfhydroxyl groups are bonded to any group that, when administered to a subject, cleave to form a free hydroxyl, amino, or sulfhydroxyl group, respectively. Examples of prodrugs include, but are not limited to, acetate, formate, benzoate and phosphate ester derivatives of hydroxyl functional groups, especially the hydroxyl group on the phenyl ring of formula I, and acetyl and benzoyl derivatives of amine functional groups in the compounds of the invention and the like.

The compounds of the invention can exist in unsolvated as well as solvated forms, including hydrated forms. In general, the solvated forms, including hydrated forms and the like are equivalent to the unsolvated forms for purposes of the invention.

The compounds are of course given by forms suitable for each administration route. For example, they are administered in drops, tablets or capsule form, by injection, inhalation, eye lotion, ointment, foams, suppository, etc. by topical, vaginal or rectal administration. Parenteral or topical administration is preferred. The compounds of the invention are useful for the treatment of infections in hosts, especially mammals, including humans, in particular in humans and domesticated animals. The compounds may be used, for example, for the treatment of infections of skin, mouth, the respiratory tract, the urinary/reproductive tract, and soft tissues and blood, especially in humans. In one embodiment of the invention diseases are those caused by or associated with infection by microorganisms including, but are not limited to, Streptococcus pyogenes, Staphylococcus aureus, methicillin resistant Staphylococcus aureus (“MRSA”), Staphylococcus epidermidis, Bacillus anthracis, Neisseria gonorrhoeae, Neisseria meningitides, Mycobacteria tuberculosis, vancomycin resistant Enterococcae (“VRE”), Helicobacter pylori, Chlamydia pneumoniae, Chlamydia trachomatis, Campylobacter jejuni, Propionibacterium acnes, Pseudomonas aeruginosa, Haemophilus influenzae, Streptococcus pneumoniae, Enterococcus faecalis, Escherichia coli, Corynebacterium diphtheriae, Morazella catarrhalis and Bacillus cereus.

The pharmaceutical compositions of the present invention employ the compounds of the invention, and may include inert, pharmaceutically acceptable carriers that are either solid or liquid. Solid form compositions include powders, tablets, dispersible granules, capsules, cachets and suppositories. A solid carrier can be one or more substances which may also act as diluents, flavoring agents, solubilizers, lubricants, suspending agents, binders, or tablet disintegrating agents; it can also be an encapsulating material. In powders, the carrier is a finely divided solid which is an admixture with the finely divided active compound. In the tablet the active compound is mixed with carrier having the necessary binding properties in suitable proportions and compacted in the shape and size desired. The powder and tablet preferably contain from 5 to about 70 percent and preferably 10 to about 60 percent of the active ingredient. Suitable solid carriers are magnesium carbonate, magnesium stearate, talc, sugar, lactose, pectin, dextrin, starch, gelatin, tragacanth, methyl cellulose, sodium carboxymethyl cellulose, a low melting wax, cocoa butter, and the like. The term “preparation” is intended to include compositions wherein the formulation of the active compound with encapsulating material acting as carrier. This provides a capsule in which the active component (with or without other carriers) is surrounded by a carrier, which is accordingly in association with it. Similarly, cachets are included. Tablets, powders, cachets, and capsules can be used as solid dosage forms suitable for oral administration.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils, glycerol, polyethylene glycols and fatty acid esters of sorbitan, and mixtures thereof. Aqueous solutions suitable for oral use can be prepared by dissolving the active component in water and adding suitable colorants, flavors, stabilizing, and thickening agents as desired. Aqueous suspensions suitable for oral use can be made by dispersing the finely divided active component in water with viscous material, i.e. natural or synthetic gums, resins, methyl cellulose, sodium carboxymethyl cellulose, and other well-known suspending agents.

An example, for instance, is water or water-propylene glycol solutions for parenteral injection. Such solutions are prepared so as to be acceptable to biological systems (isotonicity, pH, etc). Liquid preparations can also be formulated in solution in aqueous polyethylene glycol solution.

Formulations of the present invention which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery over time of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

The compositions of the invention may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

A physician or veterinarian having ordinary skill in the art can readily determine and prescribe the effective amount of the pharmaceutical composition required. For example, the physician or veterinarian could start doses of the compositions of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, topical, intravenous and subcutaneous doses of the compositions of this invention for a patient, when used for the indicated effects, will range from about 0.0001 to about 100 mg per kilogram of body weight per day, more preferably from about 0.01 to about 50 mg per kg per day, and still more preferably from about 0.1 to about 10 mg per kg per day. Each unit dose may be, for example, 5, 10, 25, 50, 100, 125, 150, 200 or 250 mg of the compound of the invention. If desired, the effective daily dose of the active compound may be administered as two, three, four, five, six or more sub-doses administered separately at appropriate intervals throughout the day, optionally, in unit dosage forms.

Synthesis

The compounds of the present invention may be synthesized according to the chemistry outlined in the following schemes. It will be apparent to those skilled in the art that the described synthetic procedures are merely representative in nature and that alternative procedures are feasible and may be preferred in some cases.

The chemistry to synthesize diaryl ethers by the cross-coupling reaction of an aryl halide with a phenol derivative is well known to those skilled in the art of organic chemistry and has been well documented recently by 1) Theil, F, Angew. Chem. Int. Ed. 1999, 38, 2345; 2) Sawyer, J. S., Tetrahydron, 2000, 56, 5045; 3) Ley, S. V. et al, Angew. Chem. Int. Ed. 2003, 42, 5400. Many of the synthetic methodologies and reactions can be employed to the preparation of precursors, possessing 2-aryloxyphenol core structure and their hydroxyl-protected analogs, of the compounds in the invention of formula I.

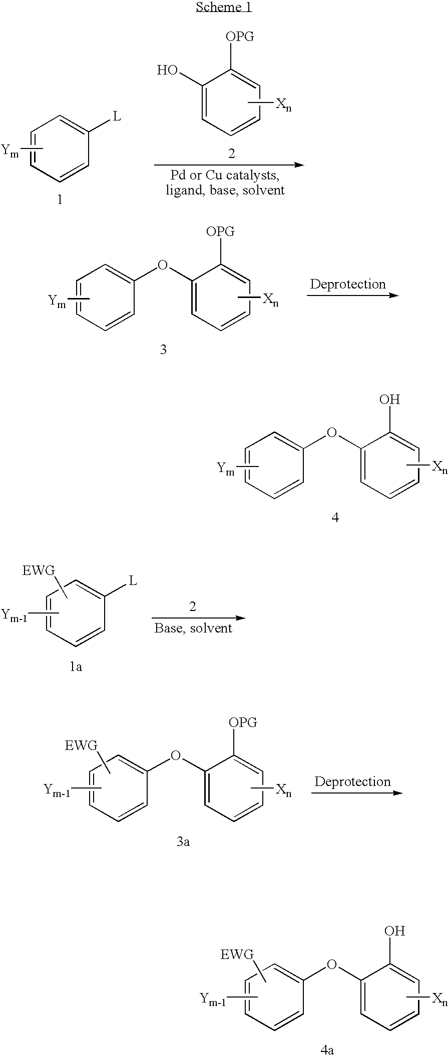

As shown in Scheme 1, the cross-coupling reaction of aryl halide 1 with mono-protected catecol 2, in the presence of a palladium or copper catalyst combined with a suitable ligand and a base at elevated temperature, affords 3. The catalyst suitable for the reaction is Pd(OAc)₂, Pd(dba)₂, Cu₂O, CuI, CuCl or (CuOTf)₂.C₆H₆ etc. and the ligand is PPh₃, 2,2′-bis(diphenylphosphino)-1,1′-binaphtyl (BINAP), 1,1′-bis(diphenylphosphino)ferrocene (DPPf), P(t-Bu)₃, N,N-dimethylglycine, 1-naphthoic acid or 2,2,6,6-tetramethylheptane-3,5-dione and the like. The base used in the reaction is Cs₂CO₃, K₂CO₃, Na₂CO₃, sodium or potassium (tert)-butoxide. Solvents which may be used in the reactions include THF, 1,4-dioxane, toluene, N,N-dimethylacetamide (DMA), N,N-dimethylformamide (DMF), acetonitrile (ACN) and the like. The cross-coupling reaction proceeds at 50° C.-150° C., optionally with assistance of ultrasonic or microwave irradiation. Alternatively, if 1 bears electron withdrawing groups (EWG) such as CN, CHO, NO₂, CO₂Me etc. at para- or ortho-position of the halide (such as F or Cl) the reaction proceeds through S_{N}Ar mechanism, without the need of any catalyst and ligand. Deprotection of 3 (including 3a) gives corresponding hydroxyl free compounds 4 including 4a). Most commercial or literature mono-protected catecols 2 bear methyl, benzyl, or silyl group as hydroxyl protection groups. Such protection groups are easily removed from 3 (including 3a) with acids, contact hydrogenation, BBr₃, BI₃, MgI₂, NaSEt and tetrabutylammonium fluoride etc. in protic or aprotic solvents at −78° C.-100° C.

Intermediate 4 (including 4a) is converted into the compounds in the invention of Formula I through corresponding 4-halo, cyano or amino derivatives, 5, 6 and 8, which are prepared by the procedures well known for those skilled in the art. The synthesis of 5 and 7 becomes possible because of the extraordinary reactivity and regioselective chemistry of the phenolic benzene ring toward electrophilic substitution reactions at the position-4. As shown in Scheme 2, halogenation of 4 with N-bromo-succinimide (NBS) or iodochloride provides halide 5 while nitration of 4 with nitric acid gives compound 7, which is subsequently converted into amine 8 by the reduction with tin (II) chloride. Cyano compound 6 is obtained by the reaction of 5 with CuCN at elevated temperature.

Schemes 3-6 illustrate how compounds 5, 6 and 8 are transformed to the compounds in the present invention.

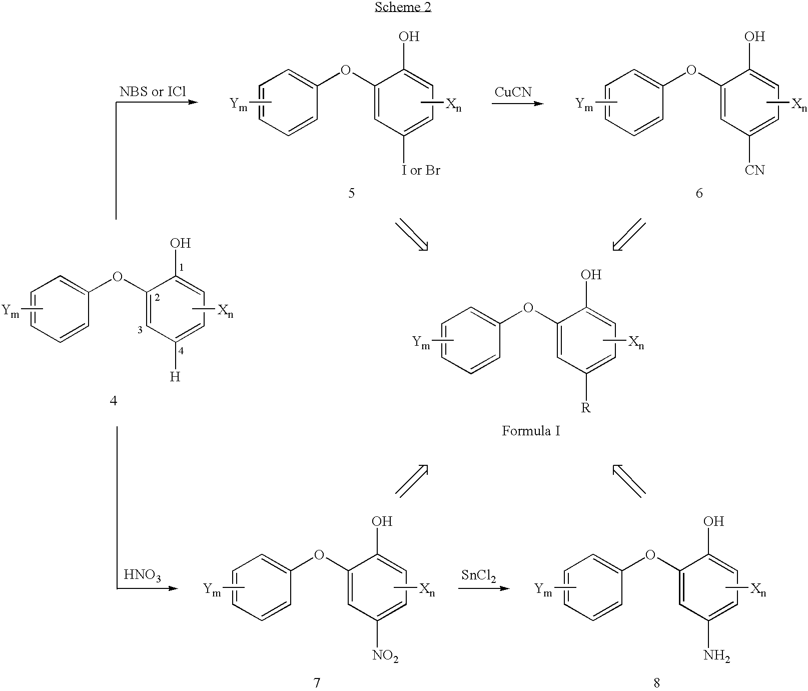

The conversion of halides 5 into the compounds of the present invention is shown in Scheme 3 below. Boronic acid of the present invention (formula I wherein R is —B(OH)₂), 9, is made by the procedure outlined in Org. Synth. Coll., 4, 68 (1963). Thus the aryllithium generated by the halo-metal exchange of 5 with butyl lithium at −78° C. is quenched by trimethyl borate, forming corresponding arylborate, which is then hydrolyzed to corresponding boronic acid.

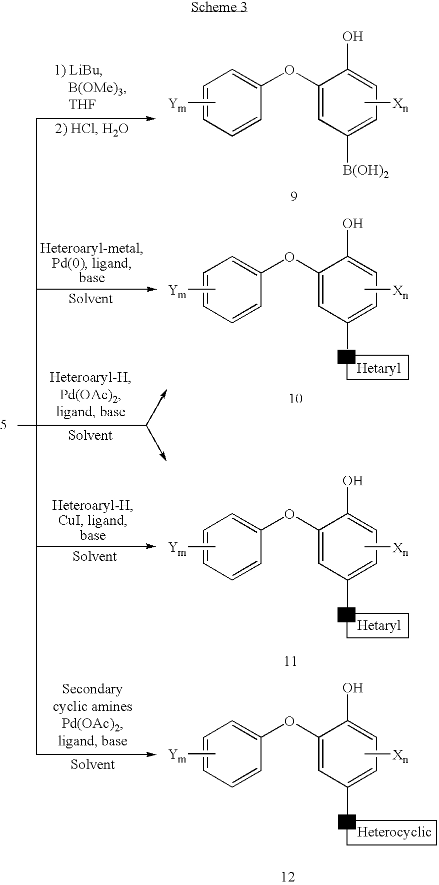

As described in Scheme 3, 5 can undergo transition-metal catalyzed cross-coupling reactions with heterocyclic agents, giving the compounds of the present invention, 10-12. The reactions are well known to those skilled in the art as named reactions such as Suzuki coupling, Stille coupling, Negishi coupling and the like.

Compound 10 (Formula I, wherein R═R¹=(a), (b), (c)-(i), (f)-(i) and (ii), (i)-(i) and (ii) and the like) is attained by the reaction of 5 with heteroaryl-metal agents, such as heteroarylB(OH)₂, heteroarylSn(Bu)₃ and heteroarylZnCl, or in some cases directly with heteroaryl-H, under the conditions outlined by K. J. Stille (Angew. Chem. Int. Ed. Engl. 1986, 25, 508-524), A. Suzuki (J. Organomet. Chem. 1999, 576, 147-168), T. Balle (J. Med. Chem. 2003, 46, 265) and B. Sezen (J. Am. Chem. Soc. 2003, 125, 5274-5275) by the formation of a C—C bond between phenyl ring and heteroaryl moiety.

Compound 11 (formula I, wherein R═R¹=(c)-(iii), (f)-(iii), (i)-(iii) and the like) is obtained by the reaction of 5 with heteroaryl-H by procedures outlined by A. Klapars (J. Am. Chem. Soc. 2001, 123, 7727-7729) and J. C. Antilla (J. Org. Chem. 2004, 69, 5578-5587) by the formation of a C—N bond between phenyl ring and heteroaryl moiety.

Compound 12 (Formula I, wherein R═R³=morpholin-4-yl, 4-methyl-piperazin-1-yl etc.) is made from 5 by the cross-coupling reaction with cyclic secondary amines according to the methods outlined by S. L. Buchward (Top. Curr. Chem. 2002, 219, 131) and J. F. Harwig (Handbook of Organopalladium Chemistry for Organic Synthesis; Negish, E., Ed.; Wiley-Interscience: New York, 2002; p 1051) by the formation of a C—N bond.

Heteroaryl-H, heteroarylB(OH)₂, heteroarylSn(Bu)₃ and cyclic secondary amines are literature or commercially available. HeteroarylZnCl is well known in the art and may be prepared in situ from corresponding heteroaryl-halides by following the literature, for instance, T. Balle (J. Med. Chem., 2003, 46, 265). Transition metal catalysts used in the reactions comprise Pd(Ph₃P)₄, Pd(dba)₂, Pd₂(dba)₃, Pd(OAc)₂, Cu₂O, CuI, CuCl and the like. Bases suitable for the reactions include equilibrating bases such as Na₂CO₃, K₂CO₃, Cs₂CO₃, K₃PO₄, Na₃PO₄, MgO, CaO and reversible base such as NaOtBu and KO^{t}Bu. The ligands employed in the reactions comprise PPh₃, 2,2′-bis(diphenylphosphino)-1,1′-binaphtyl (BINAP), 1,1′-bis(diphenylphosphino)ferrocene (DPPf), P(o-tolyl)₃, P(t-Bu)₃, biphenyl-2-yl-di(tert-butyl)-phosphane, di(tert-butyl)-(2′-methyl-biphenyl-2-yl)-phosphane, [2′-(di(tert-butyl)-phosphanyl)-biphenyl-2-yl]-dimethyl-amine, trans-1,2-cyclohexanediamine and the like. Solvents used in the reactions include THF, 1,4-dioxane, toluene, DMA, DMF and the like. The cross-coupling reaction proceeds at 50° C.-150° C.

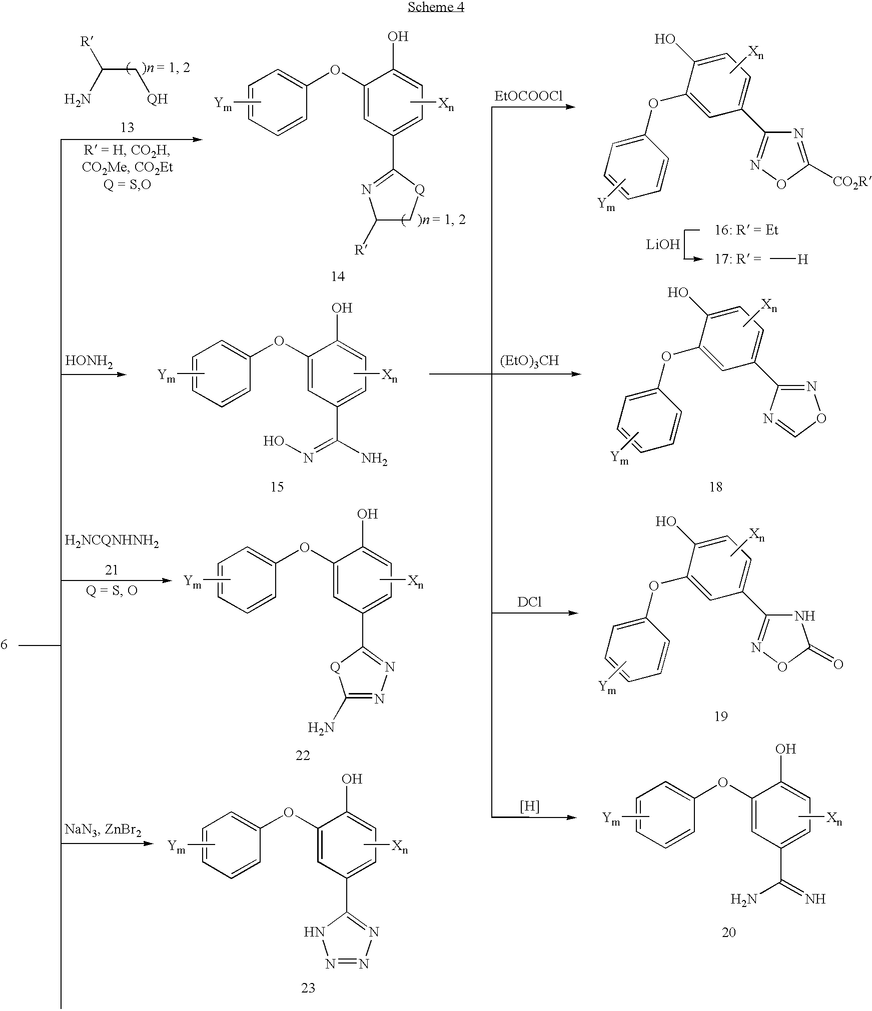

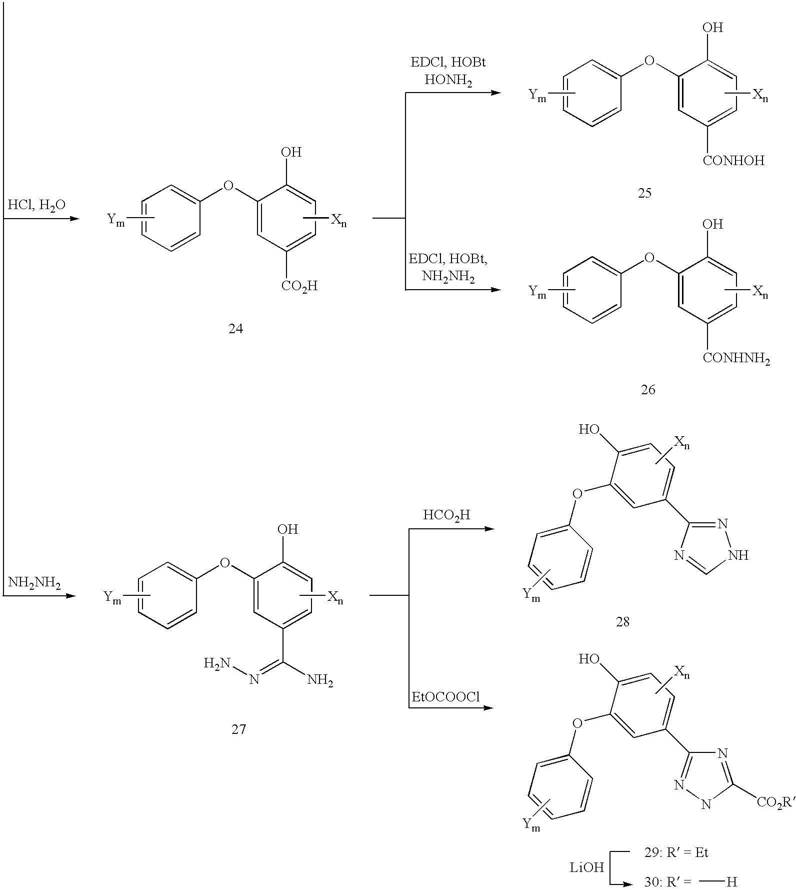

Alternatively, the compounds of the present invention, which have a C—C bond linked between R and the position 4 of the phenolic ring of formula I, are made from 6 by the reactions of the cyano group with various reagents as shown in Scheme 4.

Compound 14 (formula I wherein R═R¹=(v)) is made by the reactions of 6 with β or γ-hydroxy- or mecapto-alkylamine 13 in aqueous alcohol under the conditions as described by R. J. Bergeron (J. Med. Chem. 2003, 46, 1470-1477). By reacting with hydroxylamine, 6 is conveniently converted into N-hydroxyl-amidine 15 (formula I wherein R≡C(NOH)NH₂)), which is further transformed to 1,2,4-oxadiazoles 16, 18 and 19 (formula I, R═R¹=(r)-(i) and (u)) by the cyclization with ethyl oxalyl chloride, triethyl orthoformate and carbonyl diimidazole respectively according to the methods disclosed by M. J. Genin (J. Med. Chem. 2000, 43, 953-970). Hydrolysis of ester 16 provides corresponding acid 17. Alternatively, contact hydrogenation of 15 provides amidine 20 (formula I wherein R═—C(NH)NH₂). Reactions of compound 6 with semicarbazide and thiosemicarbazide 21 in trifluoroacetic acid (TFA), by the procedure outlined by G. Chauviere (J. Med. Chem. 2003, 46, 427-440), give rise to 1,3,4-oxadiazole and thiodiazole 22 (formula I, R═R1=(p), (q)) respectively. Tetrazole derivative 23 (formula I, R═R1=(s)) are attained by the reaction of 6 with sodium azide and zinc bromide in isopropanol or dioxane at 50-120° C. as disclosed by K. B. Sharpless (Org. Lett. 2002, 4, 2525-2527). Compound 6 is hydrolyzed to acid 24 (formula I, R═—CO₂H) under standard conditions well known in the art. Acid 24 is diversified precursor for the synthesis of acid derivatives based on procedures well known in the art. For example, condensation of 24 with hydroxylamine and hydrazine in the presence of 1-ethyl-3-(3-dimethylaminopropyl)-carbodiimide (EDCI) and 1-hydroxybenzotriazole (HOBt) gives N-hydroxy amide 25 and carbazide 26 (formula I, R≡CONHOH and —CONHNH₂) respectively. Compound 27 (formula I wherein R═—C(NHNH₂)NH₂) is attained by the addition of hydrazine to 6 according to the procedure described by J. Roppe (J. Med. Chem. 2004, 47, 4645-4648), and converted to 28 and 29 (formula I, R═R¹=(o)-(iii)) by the reaction with formic acid and ethyl oxalyl chloride respectively. Hydrolysis of 29 affords acid 30.

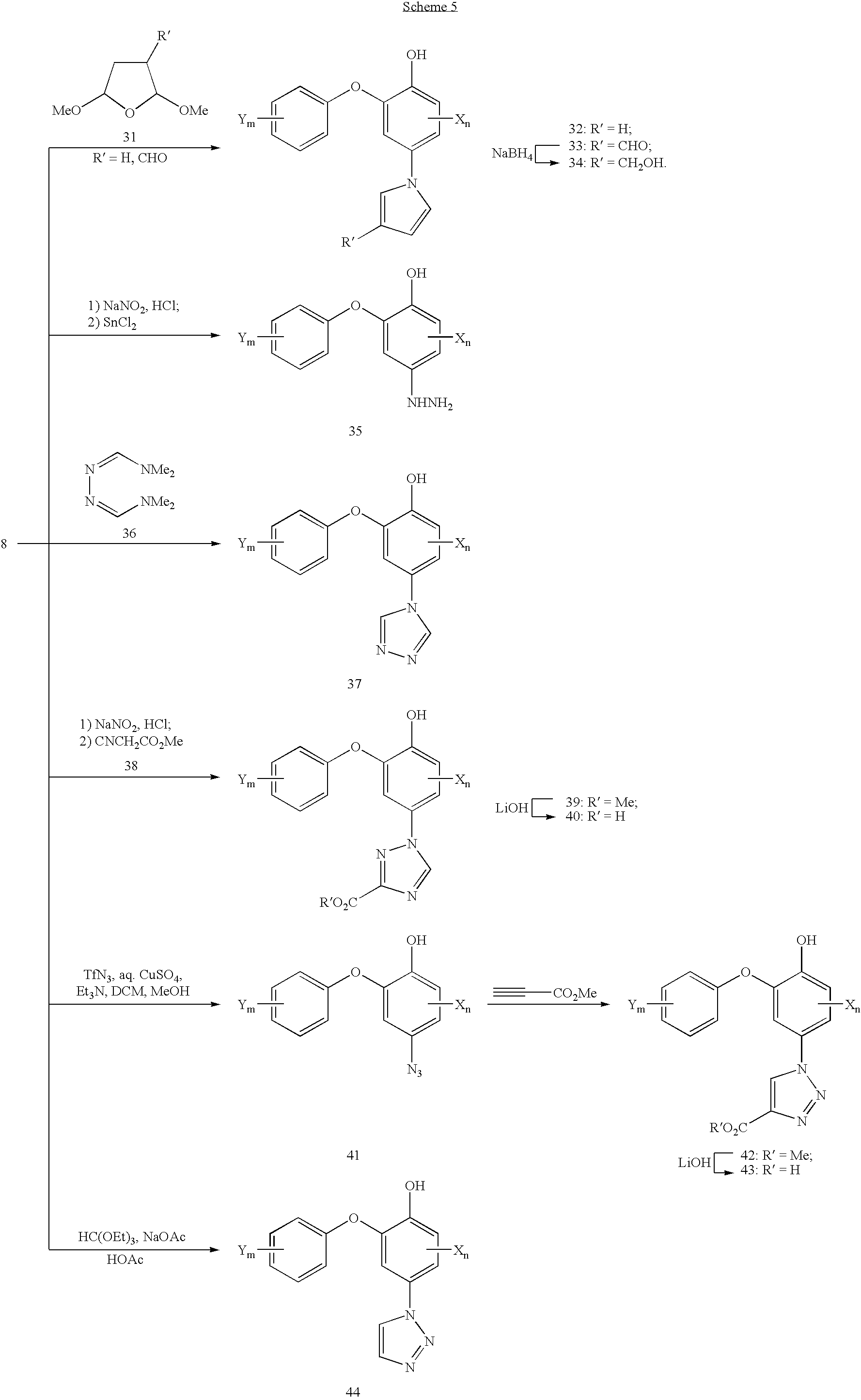

The compounds of the present invention in which there is a C—N bond linked between R and the position 4 of the phenyl ring are synthesized from amine 8 as depicted in Scheme 5 according to the procedures outlined by M. J. Genin (J. Med. Chem. 2000, 43, 953-970).

Thus, pyrroles 32 and 33 (formula I, R═R¹=(c)-(iii)) are made by the condensation of 8 with reagent 31 at elevated temperature. Reduction of 33 with sodium borohydride yields alcohol 34. Diazotization of 8 with sodium nitrite and hydrochloric acid, followed by reduction with tin (II) chloride provides hydrazine 35 (formula I, R═—NHNH₂). 4H-1,2,4-Triazole derivative (formula I, R═R¹=(n)-(ii)), 37, is synthesized from 8 by the reaction with reagent 36 prepared by the method described by R. K. Bartlett (J. Chem. Soc. (C), 1967, 1664) at 30-120° C. 1H-[1,2,4]-Triazole derivatives 39 (formula I, R═R¹=(o)-(ii)) are attained by diazotization of 8, followed by 1,3-dipolar cycloaddition with methyl isocyanoacetate 36 in situ at —10-0° C. Alternatively, compound 8 is converted to azide 41 by the reaction reported by Q. Liu (Liu, Q. et al, Org. Lett., 2003, 5, 2571), using fleshly prepared triflyl azide in aqueous CuSO₄ in dichloromethane (DCM) and methanol in the presence of triethylamine at 0-50° C. 41 undergoes 1,3-dipolar cycloaddition with methyl propionate in toluene or benzene at reflux, leading to 1H-1,2,3-triazole 42 (formula I, R═R¹=(j)-(iii)). Furthermore, reaction of 8 with triethyl orthoformate and sodium acetate in acetic acid at the 60-120° C. affords tetrazole 44 (formula I, R═R¹=(t)). Hydrolysis of ester 39 and 42 performed in aqueous lithium hydroxide solution lead to acids 40 and 43, which are further converted to corresponding acid derivatives by the procedures well known to those skilled in the art.

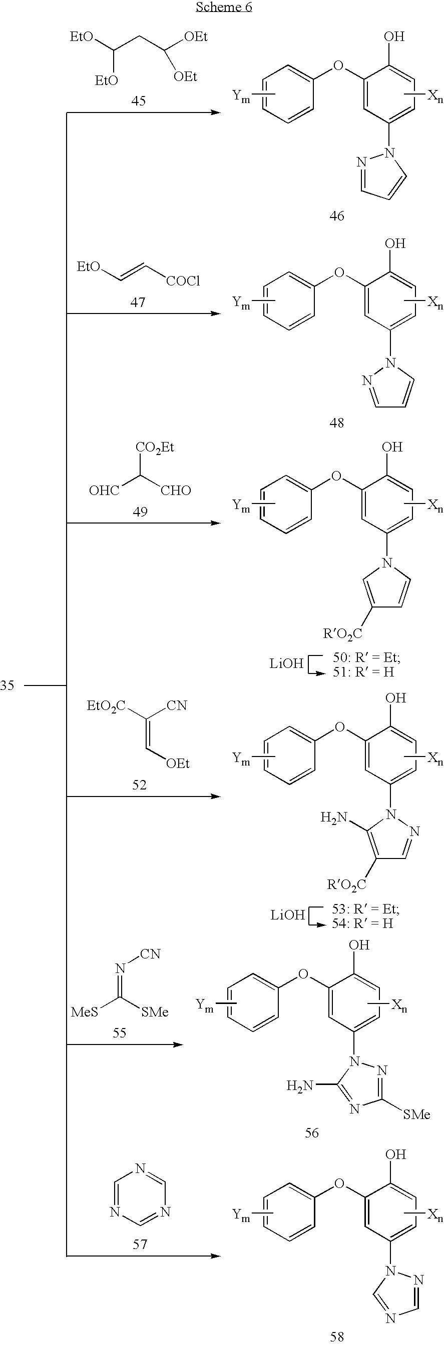

Scheme 6 outlines the methods by which hydrates 35 can be converted to other compounds of structural formula I. As shown, syntheses of pyrrazoles 46, 48, 50 and 53 (formula I, R═R¹=(f)-(iii)) are accomplished by the cyclization of 35 with reagents 45, 47, 49 and 52 respectively under the conditions described by J. Roppe (J. Med. Chem. 2004, 47, 4645-4648), M. J. Genin (J. Med. Chem. 2000, 43, 953-970) and J. Y. Hwang (J. Comb. Chem. 2005, 7, 136-141). 1H-[1,2,4]Triazoles 56 and 58 (formula I, R═R¹=(o)-(ii)) are made by the cyclization of 35 with reagents 55 and 57 respectively. Hydrolysis of esters 50 and 53 leads to acid 51 and 54 respectively. The reagents 45, 52, 55 and 57 are commercially available. The reagents 47 and 49 are prepared according to the methods outlined by L. F. Tietze (Synthesis, 1993, 1079) and S. H. Bertz (J. Org. Chem. 1982, 47, 2216-2217).

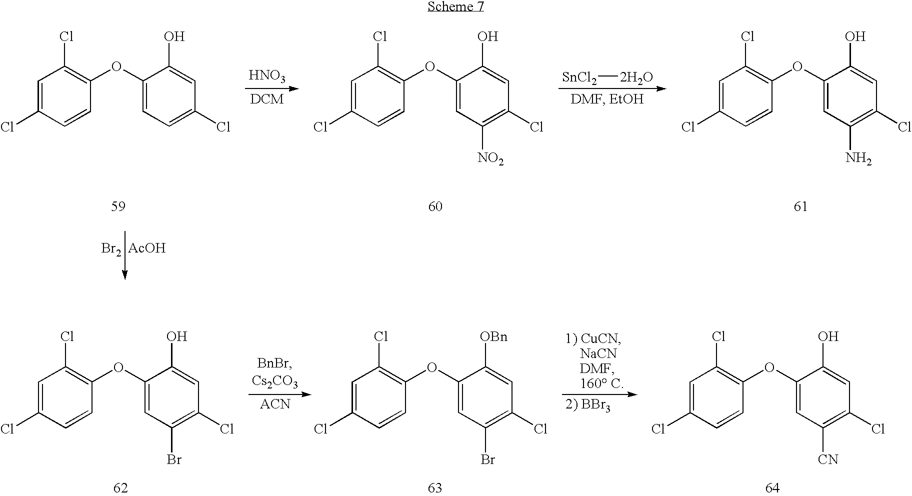

Schemes 7-12 demonstrate the syntheses of specific compounds depicted by Formula I of the present invention.

As shown in Scheme 7, nitration of commercial 5-chloro-2-(2,4-dichloro-phenoxy)-phenol (triclosan) 59 in dichloromethane by nitric acid at ambient temperature afforded 4-nitro-triclosan 60 as major product. Reduction of 60 with tin (II) chloride in DMF and ethanol provided 4-amino-triclosan 61 in good yield. Halogenation of triclosan in acidic acid with bromine gave 4-bromo-triclosan 62 predominantly. O-Benzylation of 62 by the reaction with cesium carbonate in acetonitrile led to 63, which was further cyanided with copper (I) cyanide and sodium cyanide in DMF at elevated temperature and subsequently deprotected with boron tribromide giving 4-cyano-triclosan 64. Compounds 61, 62, 63 and 64 are key intermediates for the synthesis of 4-substituted triclosan derivatives of the instant invention as shown in following schemes.

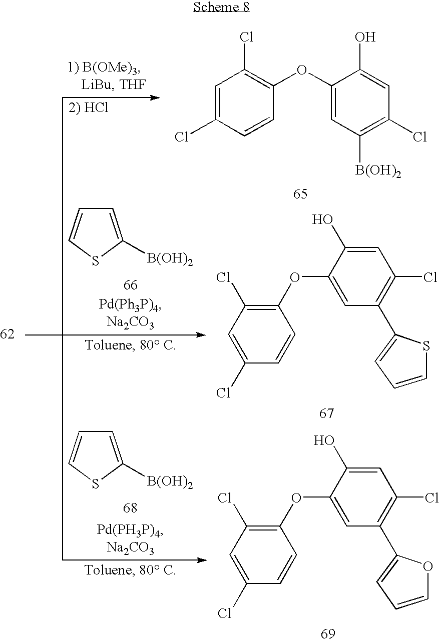

Boronic acid 65 was obtained by the reaction of bromide 62 with butyl lithium and trimethyl borate, followed by hydrolysis in hydrochloric acid. Suzuki cross-coupling of 62 with reagents 66 and 68 in the presence of sodium carbonate and catalytic amount of Pd(Ph₃P)₄ in toluene at 80° C. afforded thiophenyl triclosan 67 and furanyl triclosan 68 respectively.

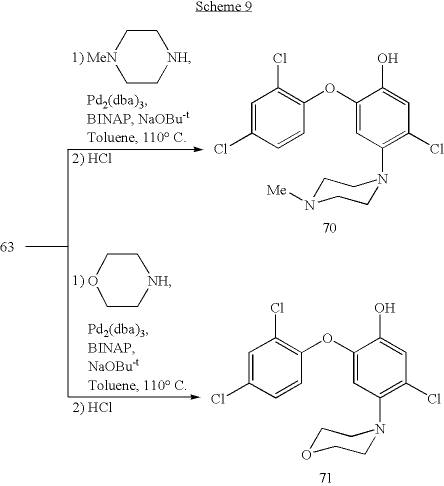

Scheme 9 describes the conversion of 63 to compounds 70 and 71, which has a saturated heterocyclic group (R³) at the position-4 of triclosan. As shown, the cross coupling reaction of 63 with 4-methyl-piperazine and morpholine proceeded in the presence of the catalyst Pd₂(dba)₃-BINAP and sodium (tert)-butoxide in toluene at 110° C., followed by removing of the O-benzyl group by hydrochloric acid and led to 70 and 71 in good yields.

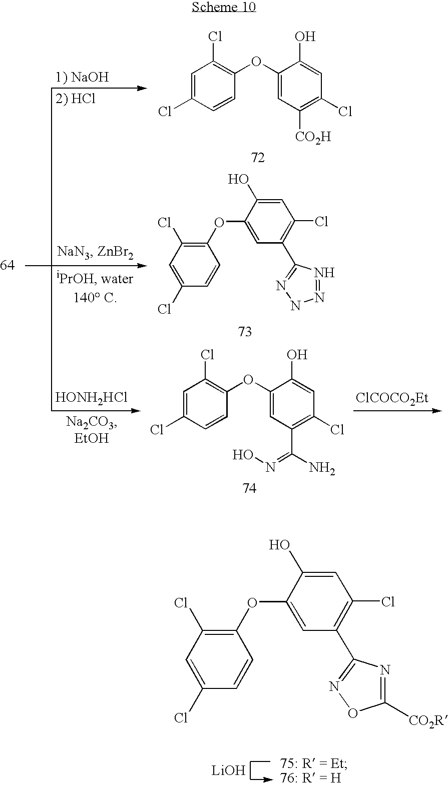

Scheme 10 outlines the reactions through which cyanide 64 was converted to the target compounds of 4-substituted triclosan. As shown, hydrolysis of 64 afforded corresponding acid 72 while the cyclo-addition with sodium azide promoted by zinc bromide in isopropanol and water at 140° C. gave tetrazole 73 exclusively. Addition of hydroxylamine to 64 was accomplished in ethanol at reflux temperature leading to 74, which was further cyclized with ethyl oxalyl chloride to oxadiazole 75. Hydrolysis of ester 75 provided acid 76.

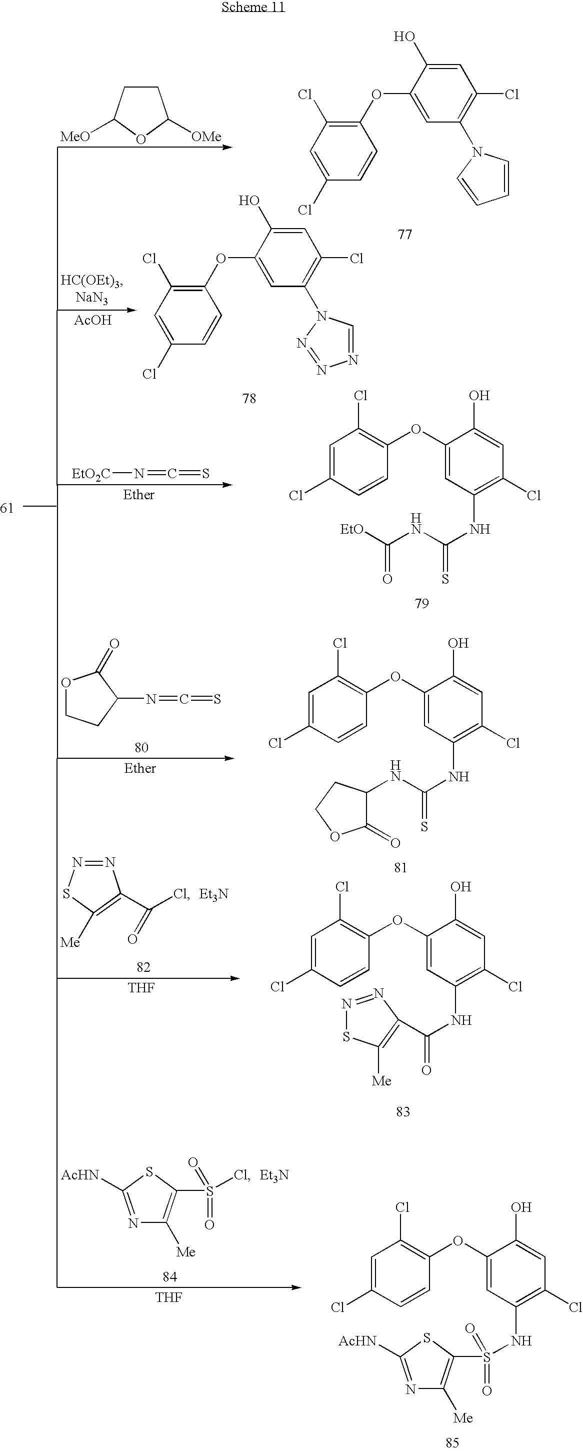

Scheme 11 describes the conversion of 61 to the triclosan derivatives possessing C—N bond linked to R. Condensation of anime 61 with 2,5-dimethoxy-tetrahydro-furan in acetic acid at 100° C. gave pyrrole 77 in good yield. Alternatively, Reaction of 61 with thiethyl orthoformate and sodium azide in refluxed acetic acid provided tetrazole 78 as the only product. Thiourea derivatives 79 and 81 were attained by the addition of 61 to isothiocyanatoformate and 80 in ether at ambient temperature. Alternatively, acylation of 61 with acid chloride 82 and 84 in the presence of triethylamine afforded amide 83 and sulfamide 85 respectively.

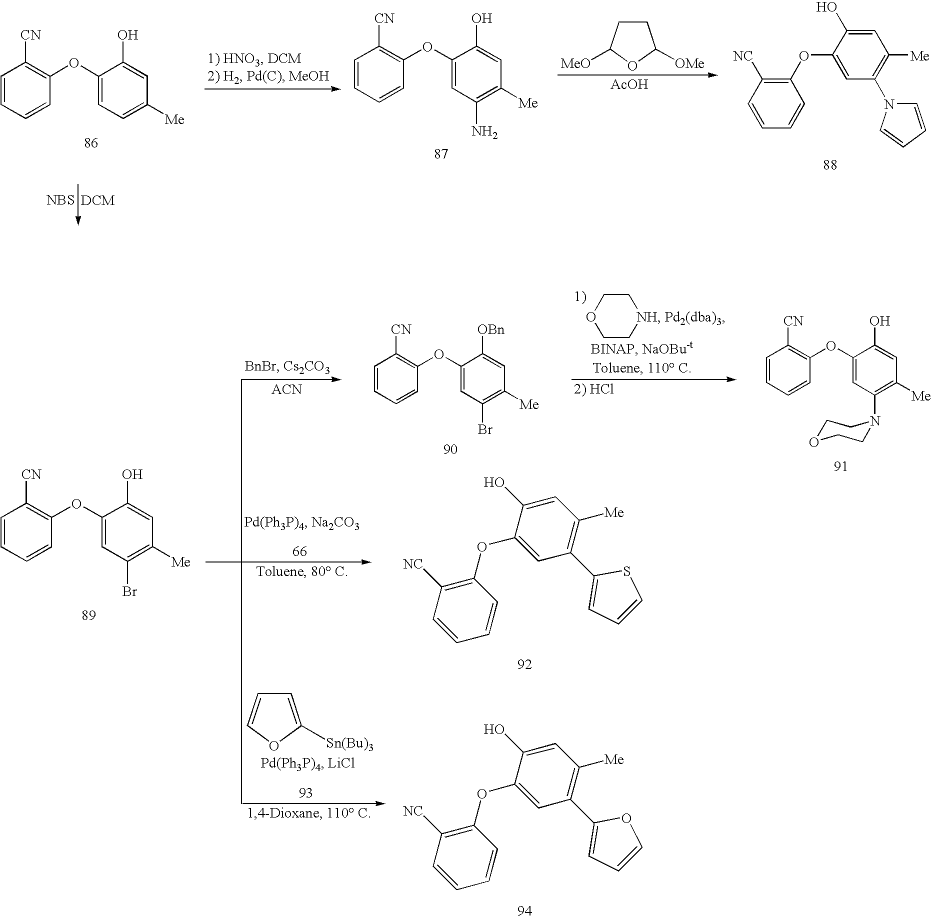

Scheme 12 demonstrates another example of the conversion of an aryloxyphenol derivative into corresponding 4-substituted derivatives of the present invention. Aryloxyphenol 86 was converted to amine 87 by nitration with nitric acid and subsequently reduction by hydrogenation. Further transformation of 87 to pyrrole 88 was carried out by the reaction with 2,5-dimethoxy-tetrahydro-furan. Alternatively, bromination of 86 with N-bromosuccinimide in dichloromethane gave 89, which was protected by the benzylation with benzyl bromide and cesium carbonate in acetonitrile, led to 90. Cross-coupling between 90 and morpholine promoted by trans-metal catalyst gave rise to 91 in good yield. Alternatively, 89 was converted to 92 by Suzuki coupling with boronic acid 66 and to 94 by Stille coupling with organotin reagent 93. The reagents maintained in Scheme 7-12 are commercially available.

### EXAMPLES

The following specific examples are provided for the purpose of further illustration only and are not intended to limit the disclosed invention.

### Example 1

### 5-Chloro-2-(2,4-dichlorophenoxy)-4-morpholin-4-yl-phenol

Step 1: 4-Bromo-5-chloro-2-(2,4-dichloro-phenoxy)phenol

To a solution of triclosan (28.95 g, 0.10 mol) in acetic acid (30 mL) cooled on ice-water bath was added a solution of bromine (16.0 g, 0.10 mmol) in acetic acid (30 mL) slowly. The solution was stirred for 1 hour below 10° C., then 3 hours at room temperature. The mixture was diluted with water (100 ml), extracted with ethyl acetate (100 ml×3), washed with saturated aqueous sodium bicarbonate solution (100 ml×3), dried over magnesium sulfate. The dry agent was removed by filtration and the filtrate was evaporated under reduced pressure. The Example title compound (i.e., 5-Chloro-2-(2,4-dichlorophenoxy)-4-morpholin-4-yl-phenol) was obtained as a white solid (35.9 g, 98%): M.P. (melting point): 79-81° C.; C₁₂H₆BrCl₃O₂ (368.44): GC-MS (gas chromatography-mass spectrometry) (EI+) m/e: 368. ¹H-NMR spectrum (proton-nuclear magnetic resonance spectroscopy) of the product was consistent with the structure anticipated.

Step 2: Benzyl 4-bromo-5-chloro-2-(2,4-dichloro-phenoxy)phenyl ether

A suspension of 4-bromo-5-chloro-2-(2,4-dichloro-phenoxy)phenol (35.9 g, 97.6 mmol), benzyl bromide (21.7 g, 127 mmol) and cesium carbonate (41.4 g, 127 mmol) in DMF (dimethylformamide) was stirred at room temperature for 4 hours. The starting material was consumed completely as revealed by TLC (thin layer chromatography). The mixture was diluted with ethyl acetate (500 ml), washed with water (200 ml×3), dried over sodium sulfate. The drying agent was removed by filtration and the filtrate was evaporated under reduced pressure. The Example title compound was obtained as a white solid (44.0 g, 98%): M.P.: 86-88° C.; C₁₉H₁₂BrCl₃O₂ (458.56): GC-MS (EI+) m/e: 458. ¹H-NMR spectrum of the product was consistent with the structure anticipated.

Step 3: 5-Chloro-2-(2,4-dichlorophenoxy)-4-(morpholin-4-yl)-phenol

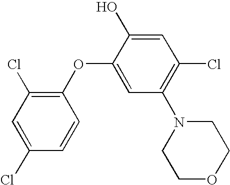

A mixture of benzyl 4-bromo-5-chloro-2-(2,4-dichloro-phenoxy)phenyl ether (13.8 g, 30.0 mmol), morpholine (3.40 g, 30 mmol), sodium tert-butoxide (4.04 g, 42 mmol), BINAP (bis(diphenylphosphino))(935 mg, 1.5 mmol), Pd₂(dba)₃ (palladium dibenzylideneacetone) (687 mg, 0.75 mmol) in toluene (30 ml) was stirred under argon at 110° C. for 5 hours. The solvent was removed under reduced pressure and the residue was stirred in refluxing acetic acid (50 ml)/concentrated HCl (50 ml) under argon for 4 hours. The mixture was diluted with water (250 ml), extracted with ethyl acetate (250 ml×3), dried on magnesium sulfate, filtered and evaporated. The crude residue was purified by flash chromatography (silica gel, ethyl acetate/hexanes=1:3). The Example title compound was obtained as an off-white solid (8.31 g, 74%): M.P.: 187-188° C.; C₁₆H₁₄Cl₃NO₃+H (374.0117): HRMS (ES+) m/e: 374.0114. ¹H-NMR (high resolution mass spectrometry) spectrum of the product was consistent with the structure anticipated.

### Example 2

### 5-Chloro-2-(2,4-dichloro-phenoxy)-4-(4-methyl-piperazin-1-yl)-phenol

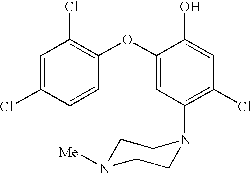

The Example title compound was synthesized by following the same procedure as described in Example 1, Step 3 with 1-methylpiperazine instead of morpholine. Rf (MeOH:dichloromethane=10%): 0.40; C₁₇H₁₇Cl₃N₂O₂+H (387.0434): HRMS (ES+) m/e: 387.0439. ¹H-NMR spectra of the product was consistent with the structure anticipated.

### Example 3

### 5-Chloro-2-(2,4-dichloro-phenoxy)-4-thiophen-2-yl-phenol

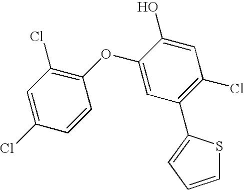

A stirred mixture of 4-bromo-5-chloro-2-(2,4-dichloro-phenoxy)phenol (200 mg, 0.540 mmol) (Example 1, Step 1), thiophene-2-boronic acid (69 mg, 0.540 mmol) and sodium carbonate (137 mg, 1.300 mmol) in toluene (5 mL), ethanol (1 mL) and water (2 mL) was purged with argon for 5 minutes and then tetrakis(tripheylphosphine)palladium (25 mg) was added. The reaction mixture was stirred under argon at 80° C. for 69 hrs, and filtered through a pad of celite. The pad was rinsed with ethyl acetate (30 mL) and the combined filtrate was diluted with water (50 mL) and extracted with ethyl acetate (50 mL×2). The organic layer was dried over magnesium sulfate, filtered and evaporated. The residue was chromatographed on silica, column eluting with 10% ethyl acetate in hexane to afford the product (177 mg, 88%) as colorless oil: R_{f} (ethyl acetate:hexane=15%): 0.35; C₁₆H₉Cl₃O₂S (369.9389): HRMS (EI+) m/e: 369.9386. ¹H-NMR spectrum of the product was consistent with the structure anticipated.

### Example 4

### 5-Chloro-2-(2,4-dichloro-phenoxy)-4-furan-2-yl-phenol

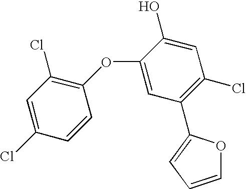

A stirred solution of 4-bromo-5-chloro-2-(2,4-dichloro-phenoxy)phenol (200 mg, 0.540 mmol) (Example 1, Step 1), furan-2-boronic acid (60 mg, 0.540 mmol) and sodium carbonate (137 mg, 1.300 mmol) in toluene (5 mL), ethanol (1 mL) and water (2 mL) was purged with argon for 5 minutes and then tetrakis(tripheylphosphine)palladium (25 mg) was added. The reaction mixture was stirred under argon at 80° C. for 24 hours, filtered through a pad of celite, which was rinsed with ethyl acetate (50 mL), and the combined filtrate was diluted with water (50 mL) and extracted with ethyl acetate (50 mL×2). The organic layer was dried over magnesium sulfate, filtered and evaporated. The residue was chromatographed on silica column eluting with 20% ethyl acetate in hexane, giving the product (21 mg, 11%) as white amorphous foam: R_{f} (ethyl acetate:hexane=15%): 0.48; C₁₆H₉Cl₃O₃ (353.9617): HRMS (EI+) m/e: 353.9615. ¹H-NMR spectrum of the product was consistent with the structure anticipated.

### Example 5

### 5-Chloro-2-(2,4-dichloro-phenoxy)-4-thiophen-3-yl-phenol (AP-268)

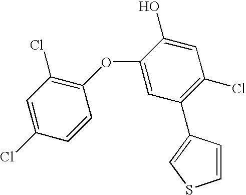

A stirred solution of 4-bromo-5-chloro-2-(2,4-dichloro-phenoxy)phenol (300 mg, 0.820 mmol) (Example 1, Step 1), thiophene-3-boronic acid (105 mg, 0.820 mmol), sodium carbonate (209 mg, 1.970 mmol), Pd₂(dba)₃ (15 mg, 0.016 mmol) and tri(o-tolyl)phosphine (29 mg, 0.096 mmol) in toluene (5 mL), ethanol (1 mL) and water (2 mL) was heated at 80° C. under argon for 2 days. The reaction mixture was filtered through a pad of celite, which was rinsed with ethyl acetate (100 mL), and the combined layer was washed with saturated ammonium chloride solution, brine and dried on magnesium sulfate. The residue was chromatographed on silica column eluting with 15% ethyl acetate in hexane giving the product (224 mg, 73%) as colorless oil. R_{f} (ethyl acetate:hexane=1:5): 0.50; C₁₆H₉Cl₃O₂S (369.9389) HRMS (EI+) m/e: 369.9395. ¹H-NMR spectrum was consistent with the structure anticipated.

### Example 6

### 2-Chloro-5-(2,4-dichloro-phenoxy)-4-hydroxy-phenylboronic acid

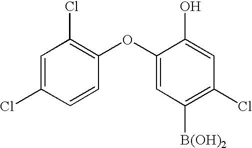

To a stirred solution of 4-bromo-5-chloro-2-(2,4-dichloro-phenoxy)phenol (500 mg, 1.360 mmol) (Example 1, Step 1) and trimethyl borate (0.305 mL, 2.72 mmol) in THF (5 mL), cooled on an acetone-ice bath, was added 2.5 M butyl lithium in hexane (1.140 mL, 2.86 mmol) dropwise via a syringe. The stirred solution was allowed to warm up to ambient temperature and left overnight. The reaction mixture was treated with 2 N HCl to pH 3, stirred for another hour and extracted with ether (25 mL×3). The organic layer was washed with brine, dried on magnesium sulfate, filtered and evaporated. The crude residue was purified by flash chromatography on silica column eluting with 35% ethyl acetate in hexane affording the title compound (205 mg, 45%) as white powder: M.P.: 180-183° C.; C₁₂H₈Cl₃O₄ (330.9503): HRMS (EI+) m/e: 330.9506. ¹H-NMR spectrum was consistent with the structure anticipated.

### Example 7

### 1-[2-chloro-5-(2,4-dichlorophenoxy)-4-hydroxyphenyl-3-(ethoxycarbonyl)thiourea

Step 1: 5-Chloro-2-(2,4-dichlorophenoxy)-4-nitrophenol

To a stirred solution of triclosan (7.24 g, 25 mmol) in dichloromethane (20 mL) was added 90% nitric acid (1.20 mL) dropwise via a syringe. The reaction mixture was stirred at room temperature until the starting material was completely consumed (30 minutes). The reaction mixture was diluted with water (100 mL), extracted with dichloromethane (100 mL×3), washed with water (100 mL), brine (100 mL) and dried with anhydrous magnesium sulfate. The drying agent was removed by filtration and the filtrate was evaporated under reduced pressure. The crude product was purified by flash chromatography (silica gel, ethyl acetate/pentane 1:5). The title compound was obtained as a light yellow solid (4.86 g, 58%): M. P.: 134-136° C.; C₁₂H₆Cl₃NO₄ (332.94): GC-MS (EI+) m/e: 335. ¹H-NMR spectrum of the product was consistent with the structure anticipated.

Step 2: 4-Amino-5-chloro-2-(2,4-dichlorophenoxy)phenol

A mixture of 5-chloro-2-(2,4-dichlorophenoxy)-4-nitrophenol (1.00 g, 2.99 mmol) and tin chloride dihydrate (3.00 g, 13.3 mmol) in DMF (2.5 mL)/ethanol (25 mL) was stirred at 70° C. under argon for 30 minutes. The reaction mixture was diluted with ethyl acetate (50 mL), neutralized with a saturated aqueous solution of sodium bicarbonate, extracted with ethyl acetate (50 mL×2), washed with water (50 mL), brine (50 mL) and dried over magnesium sulfate. The drying agent was removed by filtration and filtrate was evaporated under reduced pressure. The Example title compound was obtained as a yellow solid (911 mg, 99%): M.P.: 157-159° C.; C₁₂H₈Cl₃NO₂ (302.96): GC-MS (EI+) m/e: 305. ¹H-NMR spectrum of the product was consistent with the structure anticipated.

Step 3: 1-[2-chloro-5-(2,4-dichlorophenoxy)-4-hydroxyphenyl-3 (ethoxycarbonyl)thiourea

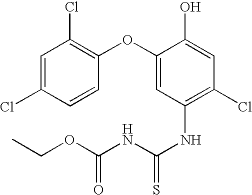

To a solution of 4-amino-5-chloro-2-(2,4-dichlorophenoxy)phenol (150 mg, 0.490 mmol) in ether (5 mL) was added ethyl isothiocyanatoformate (0.058 mL, 0.490 mmol) dropwise. After being stirred at room temperature for 1.5 hours, the reaction mixture was diluted with ether (50 mL), washed with water (20 mL), brine (20 mL) and dried over magnesium sulfate. The drying agent was removed by filtration and the filtrate was evaporated under reduced pressure. The Example title compound was obtained as a white solid (190 mg, 88%): M.P.: 170-172° C.; C₁₆H₁₃Cl₃N₂O₂ (434.9740): HRMS (EI+) m/e: 434.9724. ¹H-NMR spectrum of the product was consistent with the structure anticipated.

### Example 8

### 1-[2-Chloro-5-(2,4-dichloro-phenoxy)-4-hydroxy-phenyl]-3-(furan-2-carbonyl)-thiourea

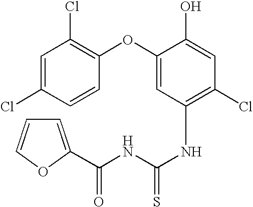

The Example title compound was made by the same procedure as described in Example 7, Step 3 with furan-2-carbonyl isothiocyanate as reagent. The product was obtained as a white solid (129 mg, 86%): M.P.: 194-196° C.

### Example 9

### 1-[4-Hydroxy-3-(2-hydroxy-phenoxy)-phenyl]-3-(ethylozycarbonyl)thiourea

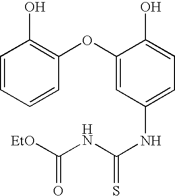

The Example title compound was synthesized by the same method as described in Example 7, Step 3, employing 4-amino-2-(2-hydroxyphenxoy)phenol as starting material. The product was obtained as a white solid (145 mg, 77%): M.P.: 136-138° C.

### Example 10

### 1-[2-Chloro-5-(2,4-dichloro-phenoxy)-4-hydroxy-phenyl]-3-(4-fluoro-phenyl)-thiourea

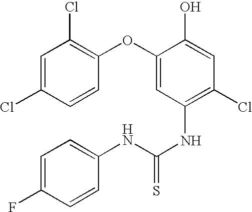

To a solution of 4-amino-5-chloro-2-(2,4-dichlorophenoxy)phenol (150 mg, 0.490 mmol) (Example 7, Step 2) in THF (tetrahydrofuran) (5 mL) was added 1-fluoro-4-isothiocyanato-benzene (75 mg, 0.490 mmol) dropwise. After being refluxed for 4 hours, the reaction mixture was diluted with ether (50 mL), washed with water (20 mL), brine (20 mL) and dried over magnesium sulfate. The drying agent was removed by filtration and the filtrate was evaporated under reduced pressure. The residue was purified by flash chromatography on silica column eluting with 25% ethyl acetate in hexane. The title compound was obtained as a white solid (143 mg, 64%): M.P.: 89-91° C.; C₁₉H₁₂Cl₃FN₂O₂S (456.9747): HRMS (EI+) m/e: 456.9759. ¹H-NMR spectrum of the product was consistent with the structure anticipated.

### Example 11

### 1-[2-Chloro-5-(2,4-dichloro-phenoxy)-4-hydroxy-phenyl]-3-(4-methoxy-phenyl)-thiourea

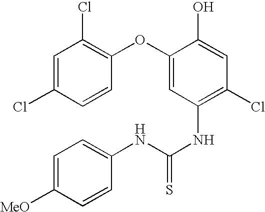

The Example title compound was made by following the procedure described in Example 11, using 4-isothiocyanato-1-methoxy-benzene as a reagent. The product was obtained as a white solid (159 mg, 69%): M.P.: 88-90° C.

### Example 12

### 1-[2-Chloro-5-(2,4-dichloro-phenoxy)-4-hydroxy-phenyl]-3-cyclohexyl-thiourea

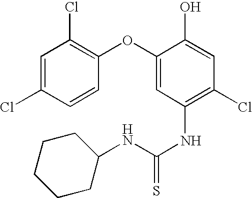

The Example title compound was made by following the procedure described in Example 11, using isothiocyanato-cyclohexane as a reagent. The product was obtained as a white powder (59 mg, 16%): M.P.: 177-179° C.

### Example 13

### 1-[2-Chloro-5-(2,4-dichloro-phenoxy)-4-hydroxy-phenyl]-3-(4-nitro-phenyl)-thiourea

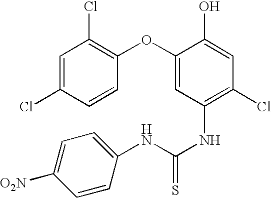

The Example title compound was synthesized by following the procedure described in Example 11, using 1-isothiocyanato-4-nitro-benzene as a reagent. The product was obtained as a yellowish amorphous foam: R_{f} (MeOH (methyl alcohol):dichloromethane=5%): 0.37. ¹H-NMR spectrum of the product was consistent with the structure anticipated.

### Example 14

### 1-[2-Chloro-5-(2,4-dichloro-phenoxy)-4-hydroxy-phenyl]-3-(2-oxo-tetrahydro-furan-3-yl)-thiourea

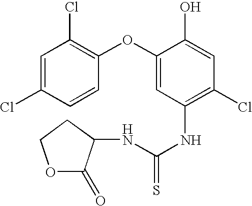

The Example title compound was made by following the procedure described in Example 11, employing 3-isothiocyanato-dihydro-furan-2-one as a reagent. The product was obtained as a white solid (128 mg, 87%): M.P.: 97-99° C.

### Example 15

### 1-(3,5-Bis-trifluoromethyl-phenyl)-3-[2-chloro-5-(2,4-dichloro-phenoxy)-4-hydroxy-phenyl]-urea

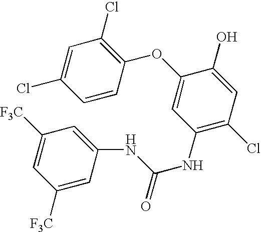

The Example title compound was synthesized by following the procedure described in Example 11, using 1-isocyanato-3,5-bis-trifluoromethyl-benzene as a reagent. The product was obtained as a white powder (126 mg, 69%): M.P.: 190-192° C.

### Example 16

### 1-(3,5-Bis-trifluoromethyl-phenyl)-3-[2-chloro-5-(2,4-dichloro-phenoxy)-4-hydroxy-phenyl]-thiourea

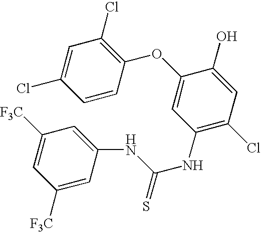

The Example title compound was synthesized by following the procedure described in Example 11, 1-isothiocyanato-3,5-bis-trifluoromethyl-benzene as a reagent. The product was obtained as a white powder (132 mg, 70%): M.P.: 157-158° C.

### Example 17

### 5-Chloro-2-(2,4-dichloro-phenoxy)-4-pyrrol-1-yl-phenol

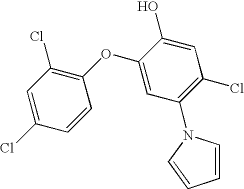

A mixture of 4-amino-5-chloro-2-(2,4-dichlorophenoxy)phenol (150 mg, 0.492 mmol) (Example 7, Step 2) and 2,5-dimethoxy-tetrahydro-furan (0.070, 0.541 mmol) in acetic acid (1 mL) was stirred at 100° C. under argon overnight. The reaction mixture was diluted with ethyl acetate (50 mL), washed with saturated sodium carbonate solution (50 mL) and subsequently brine, dried over magnesium sulfate and evaporated. The crude product was purified by flash chromatography (silica gel, ethyl acetate/hexanes 1:3). The title compound was obtained as a white powder (98 mg, 56%):M.P.: 86-87° C.; C₁₆H₁₀Cl₃NO₂ (352.9777): HRMS (EI+) m/e:352.9779. ¹H-NMR spectrum of the product was consistent with the structure anticipated.

### Example 18

### 2-(2-Hydroxy-5-pyrrol-1-yl-phenoxy)-benzonitrile

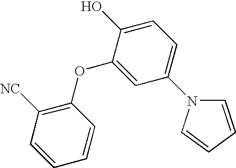

The Example title compound was obtained by following the same procedure as described in Example 19 with 4-amino-2-(2-cyanophenoxy)phenol as starting material: M.P.: 120-122° C.

### Example 19

### Thiophene-2-carboxylic acid [2-chloro-5-(2,4-dichloro-phenoxy)-4-hydroxy-phenyl]-amide

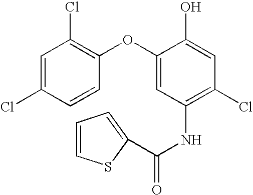

To a stirred solution of 4-amino-5-chloro-2-(2,4-dichlorophenoxy)phenol (153 mg, 0.500 mmol) (Example 7, Step 2) and triethylamine (0.097 mL, 0.700 mmol) in THF (1 mL) cooled on a ice-water bath was added dropwise a solution of thiophene-2-carbonyl chloride (0.054 mL, 0.500 mmol) in THF (1 mL). After being stirred at ambient temperature for 4 hours, the reaction mixture was evaporated under reduced pressure and the crude residue was purified by flash chromatography on silica column eluting with 25% ethyl acetate in hexane. The product (153 mg, 74%) was obtained as white amorphous foam: R_{f} (ethyl acetate:hexane=25%): 0.35; C₁₇H₁₀Cl₃NO₃S(412.9447): HRMS (EI+) m/e: 412.9453. ¹H-NMR spectrum of the product was consistent with the structure.

### Example 20

### Furan-2-carboxylic acid [2-chloro-5-(2,4-dichloro-phenoxy)-4-hydroxy-phenyl]-amide

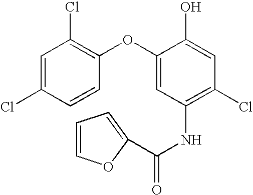

To a stirred solution of 4-amino-5-chloro-2-(2,4-dichlorophenoxy)phenol (153 mg, 0.500 mmol) (Example 7, Step 2) and triethylamine (0.097 mL, 0.700 mmol) in THF (1 mL) cooled on a ice-water bath was added dropwise furon-2-carbonyl chloride (0.050 mL, 0.500 mmol) in THF (1 mL). After being stirred at ambient temperature for 2 days, the reaction mixture was evaporated under reduced pressure and the crude residue was purified by flash chromatography on silica column eluting with 25% ethyl acetate in hexane. The product (168 mg, 84%) was obtained as white amorphous foam: R_{f} (ethyl acetate:hexane=25%): 0.35; C₁₇H₁₀Cl₃NO₄(396.9675): HRMS (EI+) m/e: 396.9679. ¹H-NMR spectrum of the product was consistent with the structure anticipated.

Examples 21-23 were synthesized by following the procedure described in Example 20, using different acid chlorides.

### Example 21

### 4-Methyl-[1,2,3]thiadiazole-5-carboxylic acid [2-chloro-5-(2,4-dichloro-phenoxy)-4-hydroxy-phenyl]-amide

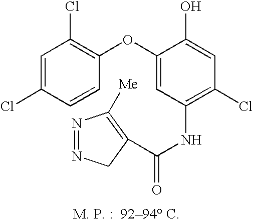

### Example 22

### 5-Methyl-isoxazole-3-carboxylic acid [2-chloro-5-(2,4-dichloro-phenoxy)-4-hydroxy-phenyl]-amide

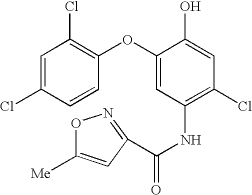

R_{f} (ethyl acetate:hexane=1:3): 0.30. ¹H-NMR spectrum of the product was consistent with the structure anticipated.

### Example 23

### N-{5-[2-Chloro-5-(2,4-dichloro-phenoxy)-4-hydroxy-phenylsulfamoyl]-4-methyl-thiazol-2-yl}-acetamide

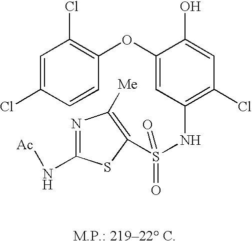

### Example 24

### 1H-Imidazole-4-carboxylic acid [2-chloro-5-(2,4-dichloro-phenoxy)-4-hydroxy-phenyl]-amide

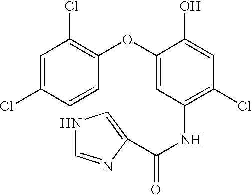

A solution of 1H-Imidazole-4-carboxylic acid (78 mg, 0.700 mmol), 4-amino-5-chloro-2-(2,4-dichlorophenoxy)phenol (214 mg, 0.700 mmol) (Example 7, Step 2), 1-(3-dimethylaminopropyl)-3-ethylcarbodiimide hydrochloride (EDCL) (148 mg, 0.770 mmol) and 1-hydroxybenzotriazole hydrate (HOBt) (104 mg, 0.770 mmol) in DCM (dichloromethane) (3 mL) and DMF (dimethylformamide) (0.3 mL) was stirred at ambient temperature under argon for 5 minutes and then triethylamine (0.351 mL, 2.520 mmol) was added. The reaction mixture was stirred at room temperature overnight, diluted with DCM (50 mL), washed with water (25 mL), saturated sodium hydrogen carbonate solution (25 mL) and dried over magnesium sulfate. The crude residue was purified by flash chromatography on silica column eluting with 10% methanol in DCM to give the title compound (38 mg, 14%) as white amorphous foam: R_{f} (methanol:chloroform=10%)::0.30; C₁₆H₁₀Cl₃N₃O₃+H (397.9866): HRMS (ES+) m/e: 397.9877. ¹H-NMR spectrum of the product was consistent with the structure anticipated.

### Example 25

### 1H-Pyrazole-4-carboxylic acid [2-chloro-5-(2,4-dichloro-phenoxy)-4-hydroxy-phenyl]-amide

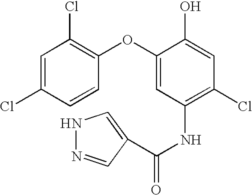

The Example title compound was synthesized by following the procedure described in Example 24, employing 1H-Pyrazole-4-carboxylic acid: R_{f} (MeOH:dichloromethane=10%). ¹H-NMR spectrum of the product was consistent with the structure anticipated.

### Example 26

### 2-(2-Hydroxy-4-methyl-5-thiophen-2-yl-phenoxy)-benzonitrile

Step 1: 2-(2-Methoxy-4-methyl-phenoxy)-benzonitrile

A suspension of 2-fluoro-benzonitrile (2.102 g, 17.36 mmol), 2-methoxy-4-methyl-phenol (2.000 g, 14.47 mmol) and cesium carbonate (5.186 g, 15.92 mmol) in DMA was vigorously stirred at 100° C. for 30 hours. The reaction mixture was poured into water, and extracted with ether (20 mL×3). The organic layer was washed with water, dried over anhydrous sodium sulfate, filtered and evaporated. The crude residue (3.452 g, 99%) was used for next reaction without further purification.

Step 2: 2-(2-Hydroxy-4-methyl-phenoxy)-benzonitrile

To a solution of 2-(2-methoxy-4-methyl-phenoxy)-benzonitrile (3.542 g, 14.43 mmol) in DCM (50 mL), cooled on an acetone-dry ice bath, was added dropwise boron tribromide (2.739 mL, 28.85 mmol) via syringe. The cooling bath was removed and the reaction was allowed to warm up to ambient temperature. After being stirred for 5 hours, the reaction mixture was quenched by adding methanol (1 mL) dropwise, followed by water (20 mL). The organic layer was separated and the aqueous layer was extracted with DCM (20 mL×3). The combined organic phase was washed with brine, dried over anhydrous sodium sulfate, filtered and evaporated. The crude solid residue was triturated in and washed with hexane, and dried in vacuum, giving the title compound (2.660 g, 82%) as white powder: M.P.: 118-119° C.; C₁₄H₁₁NO₂ (225.08): GC-MS (EI+) m/e: 225. ¹H-NMR spectrum of the product was consistent with the structure anticipated.

Step 3: 2-(5-Bromo-2-hydroxy-4-methyl-phenoxy)-benzonitrile

To a stirred solution of 2-(2-hydroxy-4-methyl-phenoxy)-benzonitrile (1.000 g, 4.440 mmol), cooled on ice-water bath, was added in portions of N-bromo-succinimide (0.831 g, 4.662 mmol). After being stirred for 10 minutes, the solvent was evaporated and the residue was subjected to flash chromatography on silica column eluting with gradient of 15-30% of ethyl acetate in hexane. The product (lower fraction, 690 mg, 51%) was obtained as white crystal: M.P.: 99-101° C.; C₁₄H₁₀BrNO₂ (302.99): GC-MS (EI+) m/e: 303. ¹H-NMR spectrum of the product was consistent with the structure anticipated.

Step 4: 2-(2-Hydroxy-4-methyl-5-thiophen-2-yl-phenoxy)-benzonitrile

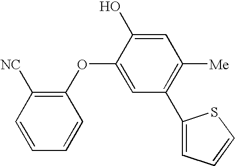

A pressure tube was charged with a mixture of 2-(5-bromo-2-hydroxy-4-methyl-phenoxy)-benzonitrile (103 mg, 0.339 mmol), 2-thiophene-boronic acid (52.0 mg, 0.407 mmol), sodium carbonate (86.24 mg, 0.812 mmol), (palladium triphenyl phosphine) Pd(PPh₃)₄ (15 mg), toluene (3 mL), EtOH (ethyl alcohol) (1 mL) and water (1 mL). After being heated and stirred vigorously at 110° C. for one day, the reaction mixture was poured into water, extracted with DCM (20 mL×3). The organic layer was washed with brine, dried over anhydrous sodium sulfate, filtered and evaporated. The crude residue was purified by column chromatography on silica gel, eluted with DCM, providing the title compound (45 mg, 43%) as white foam: R_{f} (dichloromethane): 0.35; C₁₈H₁₃NO₂S (307.0667): HRMS (EI+) m/e: 307.0673. ¹H-NMR spectrum of the product was consistent with the structure anticipated.

### Example 27

### 2-(2-Hydroxy-4-methyl-5-thiophen-3-yl-phenoxy)-benzonitrile

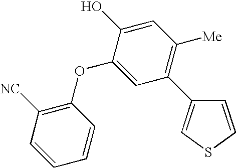

The Example title compound was obtained by following the same procedure as described in Example 27, Step 4, using 3-thiophene-boronic acid instead of 2-thiophene-boronic acid: M.P.:155-159° C.

### Example 28

### 2-(5-Furan-2-yl-2-hydroxy-4-methyl-phenoxy)-benzonitrile

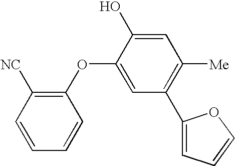

A pressure tube was charged with a mixture of 2-(5-bromo-2-hydroxy-4-methyl-phenoxy)-benzonitrile (100 mg, 0.329 mmol) (Example 26, Step 3), tributyl-furan-2-yl-stannane (129.2 mg, 0.362 mmol), anhydrous lithium chloride (42.00 mg, 0.990 mmol), Pd(PPh₃)₄ (15 mg) and 1,4-dioxane. After being heated and stirred vigorously at 110° C. for one day, the reaction mixture was poured into water, extracted with ether (20 mL×3). The organic layer was washed with brine, dried over anhydrous sodium sulfate, filtered and evaporated. The residue was purified by column chromatography on silica gel, eluted with 30% of ethyl acetate in hexane affording the title compound (80 mg, 83%) as an amorphous white foam: C₁₈H₁₃NO₃ (291.0895): HRMS (EI+) m/e: 291.0894. ¹H-NMR spectrum of the product was consistent with the structure anticipated.

### Example 29

### 2-(2-Hydroxy-4-methyl-5-pyrrol-1-yl-phenoxy)-benzonitrile

Step 1: 2-(2-Hydroxy-4-methyl-5-nitro-phenoxy)-benzonitrile

To a stirred solution of 2-(2-hydroxy-4-methyl-phenoxy)-benzonitrile (1.000 g, 4.440 mmol) (Example 26, Step 2) in DCM, cooled on an ice-water bath, was added 69% nitric acid (0.29 mL, 4.44 mmol) dropwise. The clear solution of reaction mixture turned to yellowish suspension after being stirred for 30 minutes. The solid residue was collected by filtration and the filtrate was combined with DCM (10 mL), washed with water and dried on anhydrous sodium sulfate, filtered and evaporated to about 3 mL. The precipitate was filtered off. The combined solid residue was washed with 50% of DCM in hexane and dried in vacuum. The final product (578 mg, 48%) was obtained as yellowish powder: M.P.: 157-159° C.; C₁₄H₁₀N₂O₄ (270.0641): HRMS (EI+) m/e: 270.039; ¹H-NMR spectrum of the product was consistent with the structure anticipated.

Step 2: 2-(2-Hydroxy-4-methyl-5-pyrrol-1-yl-phenoxy)-benzonitrile

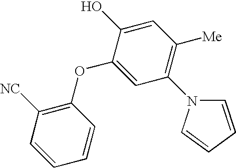

A suspension of 2-(2-hydroxy-4-methyl-5-nitro-phenoxy)-benzonitrile (100 mg, 0.370 mmol) and 10% palladium on charcoal (45 mg) in methanol was stirred vigorously in hydrogen atmosphere provided with a hydrogen balloon for 1 hour and evaporated under reduced pressure. The residue, together with 2,5-dimethoxy-tetrahydro-furan (0.053 mL, 0.407 mmol) were dissolved in acetic acid (0.25 mL), water (0.5 mL) and 1,2-dichloroethane (0.75 mL) and heated at 80° C. for 1 hour. The reaction mixture was filtered through a short column of celite, combined with water (5 mL), and extracted with DCM (10 mL×2). The organic layer was washed with water and saturated aqueous sodium carbonate solution, dried over anhydrous sodium sulfate, filtered and evaporated. The crude solid residue was triturated in 10% of DCM in hexane and dried in vacuum, giving the title compound (57 mg, 53%) as white powder: M.P.: 158-160° C.; C₁₈H₁₄N₂O₂ (290.1055): HRMS (EI+) m/e: 290.1053. ¹H-NMR spectrum of the product was consistent with the structure anticipated.

### Example 30

### 5-Chloro-2-(4-fluoro-2-hydroxy-5-morpholin-4-yl-phenoxy)-benzonitrile

Step 1: 2-(5-Bromo-4-fluoro-2-hydroxy-phenoxy)-5-chloro-benzonitrile

To a stirred solution of 2-(4-fluoro-2-hydroxy-phenoxy)-5-chloro-benzonitrile (1.000 g, 3.790 mmol) in DCM (10 mL) was added in portions of N-bromosuccinamide (0.675 g, 0.375 mmol) over a period of 1 hour. The reaction mixture was diluted with DCM (20 mL), washed with water, dried on anhydrous sodium sulfate, filtered and evaporated. The solid residue was triturated in 10% DCM in hexane and dried in vacuum giving the title compound (1.200 g, 92%) as a white powder: M.P.: 208-210° C.

Step 2: 2-(2-Benzyloxy-5-bromo-4-fluoro-phenoxy)-5-chloro-benzonitrile

To a stirred suspension of 2-(5-bromo-4-fluoro-2-hydroxy-phenoxy)-5-chloro-benzonitrile (0.500 g, 1.460 mmol), tetrabutyl ammonium iodide (10 mg), cesium carbonate (801 mg, 2.435 mmol) in acetonitrile (3 mL) was added benzyl bromide dropwise. After being stirred for 6 hours, the reaction mixture was quenched with water and extracted with DCM (20 mL×3). The organic layer was washed with water and dried on anhydrous sodium sulfate, filtered and evaporated. The solid residue was washed with hexane and dried in vacuum, affording the product (520 mg, 82%) as a white powder: M.P.: 112-210° C.

Step 3: 2-(2-Benzyloxy-4-fluoro-5-morpholin-4-yl-phenoxy)-5-chloro-benzonitrile

A pressure bottle was charged with 2-(2-benzyloxy-4-fluoro-5-morpholin-4-yl-phenoxy)-5-chloro-benzonitrile (324.2 mg, 0.749 mmol), palladium acetate (11 mg, 0.0049 mmol)) and BINAP (44.3 mg, 0.071 mmol) and toluene 5 mL), and purged with argon for 5 min. Morpholine (0.1 mL, 1.498 mmol) and sodium tert-butoxide (130 mg, 1.353 mmol) were added and the bottle was sealed, heated and stirred at 100° C. for 5 hrs. The reaction mixture was quenched with water, extracted with ethyl acetate. The organic layer was washed with water, dried over anhydrous sodium sulfate, filtered and evaporated. The residue was purified by flash chromatography on silica column eluting with 30% ethyl acetate in hexane. The product (106 mg, 32%) was obtained as a white powder which was used for next reaction directly: M.P.: 134-135° C.

Step 4: 5-Chloro-2-(4-fluoro-2-hydroxy-5-morpholin-4-yl-phenoxy)-benzonitrile

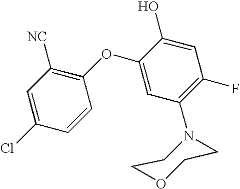

A suspension of 2-(2-benzyloxy-4-fluoro-5-morpholin-4-yl-phenoxy)-5-chloro-benzonitrile (70 mg, 0.159 mmol) and 10% palladium on charcoal in methanol was stirred vigorously in hydrogen atmosphere provided with a hydrogen balloon for 30 minutes. The reaction mixture was filtered through a short column of celite and the filtrate was concentrated. The residue was chromatographed on silica column eluting with 30% ethyl acetate in hexane. The final product (46 mg, 83%) was obtained as a white powder: M.P.: 149-151° C.; C₁₇H₁₄ClFN₂O₃+H (349.07): HRMS (ES+) m/e: 349.07. ¹H-NMR spectrum was consistent with the structure anticipated.

### Example 31

### 2-(2-Hydroxy-4-methyl-5-morpholin-4-yl-phenoxy)-benzonitrile

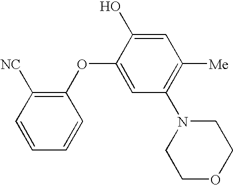

The Example title compound was obtained by following the procedures described in Example 30, Steps 2-4 with 2-(2-hydroxy-5-bromo-4-methyl-phenoxy)-benzonitrile (Example 26, Step 3) as a starting material: M.P.: 162-165° C.

### Example 32

### 5-Chloro-2-(2,4-dichloro-phenoxy)-4-tetrazol-1-yl-phenol

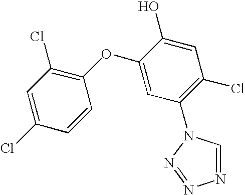

A stirred solution of 4-amino-5-chloro-2-(2,4-dichlorophenoxy)phenol (500 mg, 1.642 mmol) (Example Step 2), triethyl orthoformate (0.441 mL, 2.652 mmol) and sodium azide (160.12 mg, 2.463 mmol) in acedic acid (3 mL) was refluxed overnight. The reaction mixture was cooled to ambient temperature, diluted with water (50 mL), neutralized with sodium carbonate and extracted with ethyl acetate (20 mL×3). The organic layer was washed with brine, dried over anhydrous sodium sulfate, filtered and evaporated. The residue was purified on silica column eluting with 5% ethyl acetate in DCM. The solid residue was further triturated in hexane and dried in vacuum, affording the title compound (284 mg, 48%) as a white powder: M.P.: 147-148° C.; C₁₃H₇Cl₃N₄O₂ (356.9713): HRMS (ES+) m/e: 356.9712. ¹H-NMR spectrum of the product was consistent with the structure anticipated.

### Example 33

### 5-Chloro-2-(2,4-dichloro-phenoxy)-4-(1H-tetrazol-5-yl)-phenol

Step 1: 4-Benzyloxy-2-chloro-5-(2,4-dichloro-phenoxy)-benzonitrile

A suspension of benzyl 4-bromo-5-chloro-2-(2,4-dichloro-phenoxy)phenyl ether (459 mg, 1 mmol) (Example 1, Step 2) and copper cyanide (103 mgs, 1.15 mmol) in DMF was magnetically stirred at 160° C. for 6 hrs. 1.6 M sodium cyanide in water (25 mL) was added and aqueous layer extracted with ethyl acetate (15 mL×3). The organic layer was washed with saturated sodium bicarbonate and brine, dried over anhydrous sodium sulfate, filtered and evaporated. The crude residue (351 mgs, 87%) was used in next reaction without further purification.

Step 2: 2-Chloro-5-(2,4-dichloro-phenoxy)-4-hydroxy-benzonitrile

To a solution of 4-benzyloxy-2-chloro-5-(2,4-dichloro-phenoxy)-benzonitrile (351 mg, 0.867 mmol) in DCM (15 mL), cooled to −78° C. on an acetone/dry ice bath, was added dropwise boron tribromide (90.1 μL, 0.953 mmol) via syringe. The cooling bath was removed and the reaction was allowed to reach ambient temperature. After being stirred for 6 hours, the reaction was quenched with the addition of water (1 mL) followed by brine (20 mL). The organic layer was separated and the aqueous layer extracted with DCM (15 mL×3). The combined organic phase was washed with brine (20 mL), dried over anhydrous sodium sulfate, filtered and evaporated. The residue was purified on a silica gel column using 25% ethyl acetate/hexanes as eluent, giving the title compound (137 mg, 55%) as a white powder. M.P.: 145-150° C.; C₁₃H₆Cl₃NO₂ (312.9496): HRMS (EI+) m/e: 312.9460. ¹H-NMR spectrum of the product was consistent with the structure anticipated.

Step 3: 5-Chloro-2-(2,4-dichloro-phenoxy)-4-(1H-tetrazol-5-yl)-phenol

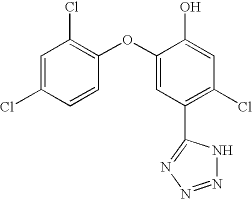

A mixture of 2-chloro-5-(2,4-dichloro-phenoxy)-4-hydroxy-benzonitrile (300 mg, 0.954 mmol), sodium azide (68.3 mg, 1.05 mmol) and zinc bromide (214.8 mg, 0.954 mmol) in 25% isopropanol/water was stirred vigorously at 140° C. for 24 hours. 6 N HCl (3 mL) and ethyl acetate (3 mL) was added to the suspension while stirring. Once mixture cleared, the layers were separated and the aqueous layer extracted with ethyl acetate (10 mL). The organic layers were combined and evaporated. The residue was dissolved in 0.25 N NaOH (15 mL) and stirred for 30 minutes at 25° C. The mixture is filtered and the solid was rinsed with 1 N NaOH (3 mL). The filtrate was acidified to pH=1, and the solid was collected and washed with 1 N HCl (3 mL) to give the title compound (283 mgs, 83%) as a white powder. M.P.: 174-180° C.; C₁₃H₇Cl₃N₄O₂ (355.9635): HRMS (EI+) m/e: 355.9640. ¹H-NMR spectrum of the product was consistent with the structure anticipated.

### Example 34

### 2-Chloro-5-(2,4-dichloro-phenoxy)-4,N-dihydroxy-benzamidine

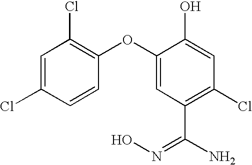

A suspension of 2-chloro-5-(2,4-dichloro-phenoxy)-4-hydroxy-benzonitrile (130 mg, 0.42 mmol) (Example 33, Step 2), hydroxylamine hydrochloride (57 mg, 0.83 mmol) and Na₂CO₃ (88 mg, 0.42 mmol) in ethanol (3 mL) and water (1.5 mL) was refluxed for 3.5 hours. The reaction mixture was evaporated under reduced pressure and residue was purified by flash chromatography employing 2.5% of methanol in DCM as eluent. The Example title compound was obtained as a white crystal (85 mg, 59%): M.P.: 137° C.; C₁₃H₉Cl₃N₂O₃ (345.97): GC-MS (CI+) m/e: 346. ¹H-NMR spectrum of the product was consistent with the structure anticipated.

### Example 35

### 3-[2-Chloro-5-(2,4-dichloro-phenoxy)-4-hydroxy-phenyl][1,2,4]oxadiazole-5-carboxylic acid ethyl ester

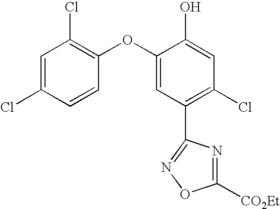

To a stirred solution of 2-chloro-5-(2,4-dichloro-phenoxy)-4,N-dihydroxy-benzamidine (70 mg, 0.202 mmol) (Example 34) in DCM (1 mL) was added dropwise pyridine (34 μL, 0.45 mmol) and subsequently ethyl oxalyl chloride (25 μL, 0.22 mmol). After being refluxed for 7 hours, the reaction was quenched with water and neutralized with 1 N HCl to pH 1 and extracted with ethyl acetate for three times. The organic layer was washed with water, dried over sodium sulfate, filtered and evaporated. The crude residue was purified by flash chromatography on silica column, eluted with 5% methanol in DCM.

The solid residue was further triturated in hexane and dried in vacuum. The final product was obtained as a white powder (54 mg, 62%): M.P.: 130° C.; C₁₇H₁₁Cl₃N₂O₅ (429.64): GC-MS (CI+) m/e: 429. ¹H-NMR spectrum of the product was consistent with the structure anticipated.

### Example 36

### 3-[2-Chloro-5-(2,4-dichloro-phenoxy)-4-hydroxy-phenyl]-[1,2,4]oxadiazole-5-carboxylic acid

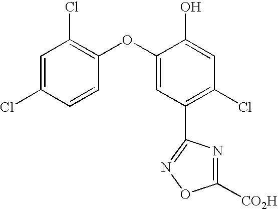

A solution of 3-[2-Chloro-5(2,4-dichloro-phenoxy)-4-hydroxy-phenyl]-[1,2,4]oxadiazole-5-carboxylic acid ethyl ester (42 mg, 0.1 mmol) (Example 35) and LiOH (8 mg, 0.3 mmol) in THF (0.5 mL) and water (0.5 mL) was stirred at room temperature for 2 hours. Water (4 mL) was added and the mixture was neutralized to pH 3 and extracted with ethyl acetate (5 mL×3). The organic layer was washed with brine, dried over sodium sulfate, filtered and evaporated. The solid residue was triturated in hexane and dried in vacuum, giving the final product as a white powder (31 mg, 89.2%): M.P.: 115° C.; C₁₅H₇Cl₃N₂O₅ (399.94): GC-MS (CI+) m/e: 356 (M⁺-CO₂). ¹H-NMR spectrum of the product was consistent with the structure anticipated.

### Example 37

### AP432: 2-Chloro-5-(2,4-dichloro-phenoxy)-4-hydroxy-benzoic acid

Step 1: 4-Benzyloxy-2-chloro-5-(2,4-dichloro-phenoxy)-benzoic acid

A suspension of 4-benzyloxy-2-chloro-5-(2,4-dichloro-phenoxy)-benzonitrile (400 mg, 0.988 mmol) (Example 33, Step 1) in ethanol (4 mL) and 10% NaOH (7 mL) was heated to 140° C. for three hours. The mixture was poured into a beaker and heated to remove excess organics. Water (10 mL) was added and the solution was acidified to pH=1 using hydrochloric acid. The solids were filtered off and washed with water and dried in vacuum. The crude material (452 mg) was used in next reaction without further purification.

Step 2: 2-Chloro-5-(2,4-dichloro-phenoxy)-4-hydroxy-benzoic acid

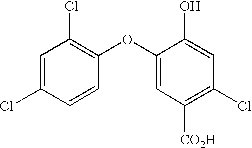

To a suspension of 4-benzyloxy-2-chloro-5-(2,4-dichloro-phenoxy)-benzonitrile (452 mg, 1.06 mmol) in DCM (6 mL), cooled to −78° C. on an acetone/dry ice bath, was added dropwise boron tribromide (120 μL, 1.27 mmol) via syringe. The cooling bath was removed and the reaction was allowed to reach ambient temperature. After being stirred for 24 hours, the reaction was quenched with the addition of saturated sodium bicarbonate solution (10 mL). The organic layer was separated and the aqueous layer extracted with DCM (15 mL×3) followed by extraction with ethyl acetate (15 mL×3). The combined organic phase was washed with brine (20 mL), dried over anhydrous sodium sulfate, filtered and evaporated. The residue was purified on a silica gel column using 30% ethyl acetate/hexanes as eluent, giving the title compound (149 mg, 42%) as a white powder. M.P.: 192-196° C.; C₁₃H₇Cl₃O₄ (331.9401): HRMS m/e: 331.9405. ¹H-NMR spectrum of the product was consistent with the structure anticipated.

Minimum Inhibitory Concentration (MIC), Broth Dilution Method:

The compounds of the present invention were tested against selected Gram positive and Gram negative organisms using standard microtitration techniques well known to those skilled in the art. Cultures of bacteria were initially applied by streaking a loopful onto agar plates under the appropriate conditions. For example, bacterial stocks are streaked for isolation of single colonies onto chocolate agar and then incubated for 18 hours at 35-37° C. in a 5% CO₂ incubator. Five to ten colonies were picked from the chocolate agar plate for subculture to Brain-Heart infusion (BHI) broth, Mueller Hinton broth, or BHI containing 4% serum, and incubated under the appropriate conditions. The ability of the test compound to act as an antimicrobial was determined by the ability of dilutions of the test substance to inhibit bacterial growth in vitro. The optical density of the culture of organisms in the presence of an active compound was compared to the optical density of the same organism grown without test compound. The activity of the compounds is described as either negative or the lowest concentration inhibiting growth (Minimum Inhibitory Concentration [MIC]).

The activity of selected compounds of the present invention against representative Gram positive and Gram negative bacteria are shown in the following Table 1.

TABLE 1
In Vitro Activity of Selected Examples of the Invention Against Bacteria

| | MIC (mg/mL) | | | | | |
|---|---|---|---|---|---|---|
| | Gram-positive Bacteria | | Gram-negative Bacteria | | Terrorism Bacteria | |
| | | S. pneumoniae | H. influenzae | | | B.anthracis |
| Example No. | S. aureus 29213 | 11733 | 43095 | E. coli 43888 | B. cereus | V770-NR1-R |
| 1 | 0.50 | 1.00 | 2.00 | 125.00 | 0.80 | 1.60 |
| 2 | 0.06 | 0.25 | 15.70 | >125 | >25 | >25 |
| 3 | 0.06 | 0.01 | 0.03 | 0.50 | 0.05 | 0.05 |
| 4 | 0.50 | 0.50 | 0.50 | 0.50 | na | na |
| 5 | 0.13 | 0.01 | 0.50 | 4.00 | 0.20 | 0.40 |
| 6 | 0.50 | na | 0.50 | 0.50 | na | na |
| 7 | 2.00 | 31.30 | 4.00 | >250 | 0.80 | 1.60 |
| 9 | 31.30 | 62.50 | 125.00 | >250 | 12.50 | 3.10 |
| 10 | 4.00 | 31.30 | 4.00 | 15.70 | 0.40 | 1.60 |
| 11 | 7.90 | 31.30 | 250.00 | >250 | na | na |
| 12 | 2.00 | 31.30 | 31.30 | >250 | na | na |
| 13 | 1.00 | 15.60 | 4.00 | >250 | na | na |
| 15 | 4.00 | na | 62.50 | >250 | 0.10 | 0.80 |
| 16 | 0.50 | na | 7.90 | >250 | 0.10 | 0.20 |
| 17 | 0.25 | >250 | 0.25 | 15.70 | 0.05 | 0.10 |
| 18 | 4.00 | 4.00 | 4.00 | >250 | >25 | >25 |
| 20 | 4.00 | 2.00 | 62.50 | >250 | >25 | >25 |
| 21 | 4.00 | 4.00 | 7.90 | >250 | na | na |
| 22 | 2.00 | 0.50 | 7.90 | >250 | na | na |
| 23 | 4.00 | 15.70 | >250 | >250 | na | na |
| 26 | 4.00 | 0.50 | 7.90 | 62.50 | 12.50 | 12.50 |
| 27 | 6.20 | >25 | 12.50 | >25 | 0.80 | 1.60 |
| 28 | 2.00 | 0.50 | 2.00 | 4.00 | 1.60 | 0.80 |
| 29 | 62.50 | 0.50 | 15.70 | 125.00 | 4.00 | na |
| 31 | 7.90 | 4.00 | 7.90 | >250 | 3.10 | 6.20 |
| 32 | 1.60 | 3.10 | 0.40 | 25.00 | 1.30 | 0.80 |
| 33 | 0.20 | na | na | >25 | 6.20 | 12.50 |
| 34 | 0.10 | na | na | 1.60 | 0.10 | 0.20 |
| 35 | 0.20 | na | na | 6.20 | 0.10 | 0.10 |
| 36 | 0.20 | na | na | 6.20 | 0.10 | 0.05 |

While the preferred embodiments of the invention have been illustrated and described, it will be appreciated that various changes can be made therein without departing from the spirit and scope of the invention.

What is claimed is:

1. A compound of Formula I or a pharmaceutically acceptable salt thereof, 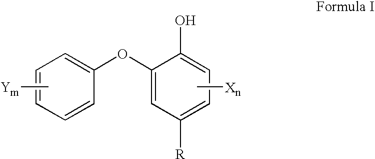
wherein,
X and Y are each halogen, CN, OH, NH₂, NMe₂, NO₂, SO₂Me, SO₃H, SO₂NH₂, CHO, CO₂NH₂, CH(NOMe), C(O)Me, CO₂Me, CO₂Et, C₁-C₄ alkyl, cycloalkyl, CF₃, SMe, OMe, or OEt;
m is 0, 1, 2, 3, 4 or 5; and
n is 0, 1, 2 or 3;
R is —B(OH)₂, —CO₂H, —CONH₂, —C(NH)NH₂, —C(NOH)NH₂, —C(NNH₂)NH₂, —C(O)NHOH, —CONHNH₂, —NHNH₂—NHC(NH)NH₂, —R¹, —NHC(O)R¹, —NHSO₂R¹, —NHSO₂R², —NHC(O)NHR², —NHC(S)NHR², [2-(2-methyl-5-nitro-imidazol-1-yl)-ethyl]oxycarbonylamino or [2-(2-methyl-5-nitro-imidazol-1-yl)-ethyl]oxycarbonyl, or —R³;
wherein R¹ is
(a) Substituted furanyl: 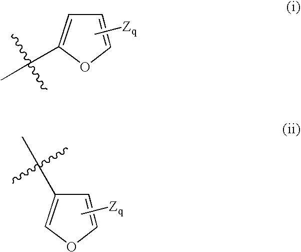
(b) Substituted thiophenyl: 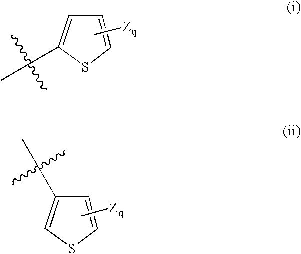
(c) Substituted pyrrolyl: 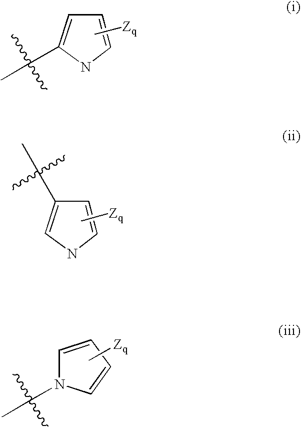
(d) Substituted isoxazolyl 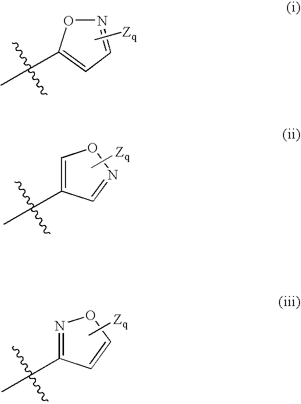
(e) Substituted isothiazolyl 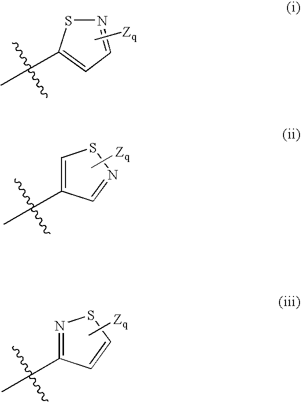
(f) Substituted pyrazolyl 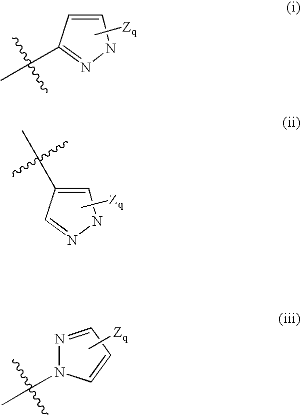
(g) Substituted oxazolyl 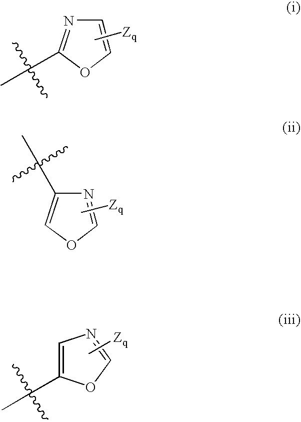
(h) Substituted thiazolyl 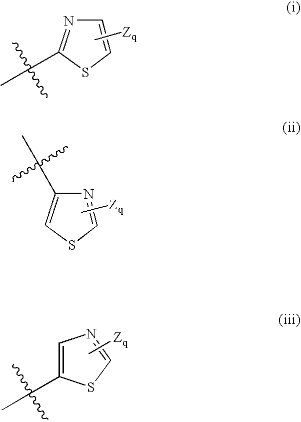
(i) Substituted imidazolyl 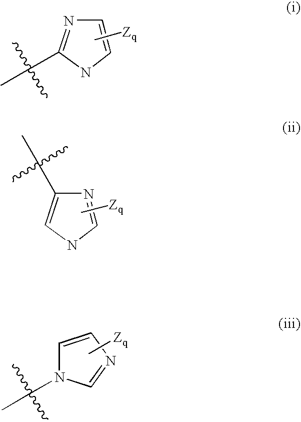
(j) Substituted 1H-[1,2,3]triazolyl 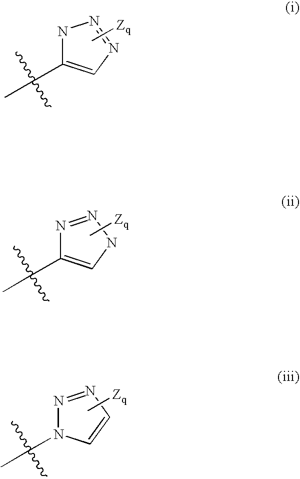
(k) Substituted 2H-[1,2,3]triazol-2-yl 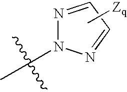
(l) Substituted [1,2,3]oxadiazolyl 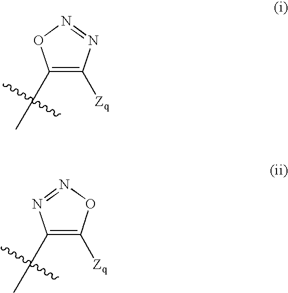
(m) Substituted [1,2,3]thiadiazolyl 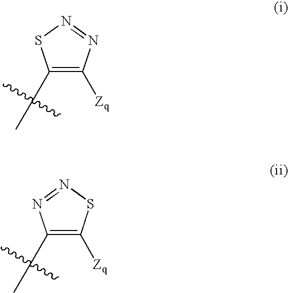
(n) Substituted 4H-[1,2,4]triazolyl 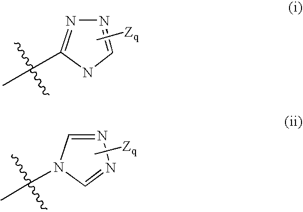
(o) Substituted 1H-[1,2,4]triazolyl 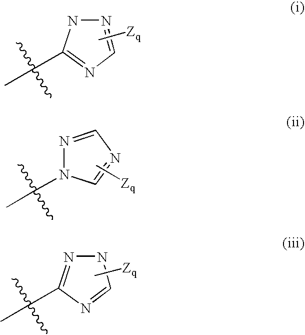
(p) Substituted [1,3,4]oxadiazolyl 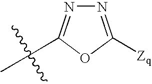
(q) Substituted [1,3,4]thiadiazolyl 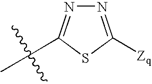
(r) Substituted [1,2,4]oxadiazolyl 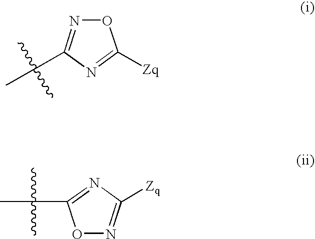
(s) 1H-Tetrazol-5-yl (i) or 2H-tetrazol-5-yl (ii) 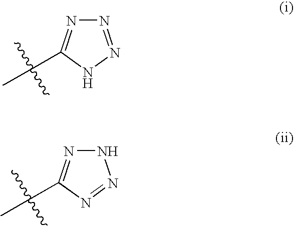
(t) 1H-Tetrazol-1-yl 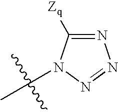
(u) 5-oxo-4H-[1,2,4]oxadiazol-3-yl 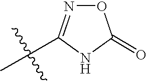
(v) Substituted 4,5-dihydro-thiazol-2-yl and 5,6-dihydro-4H-[1,3]thiazin-2-yl 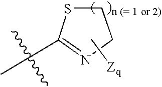
(w) Substituted pyridazinyl 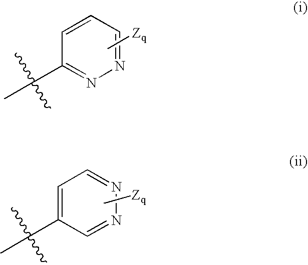
wherein Z is F, Cl, OH, NH₂, NO₂, NMe₂, NHAC, Me, Et, SMe, OMe, OEt, CHO, CN, CH₂OH, CO₂H, CONH₂, CO₂Me, CO₂Et, or SO₂Me;
q is 0, 1, 2 or 3;
R² is H, C₁-C₄ alkyl, C₃-C₇ cycloalkyl, —CO₂Me, —CO₂Et, 2-oxo-tetrahydro-furan-3-yl, 3-pyridinylcarbonyl; phenyl group substituted up to two times with F, Cl, Br, CN, OH, OMe, SMe, Me, Et, cyclopropyl, CF₃, NH₂, NMe₂, NO₂, CO₂Et, CO₂Me, CO₂H, SO₂Me, SO₂NH₂ or R³ on the ring;
wherein R³ is azetidin-1-yl, 3-amino-azetidin-1-yl pyrrolidin-1-yl, 3-amino-pyrrolidin-1-yl, 3-amino-4-methyl-pyrrolidin-1-yl, 7-amino-5-aza-spiro[2.4]hept-5-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl and thiomorpholin-4-yl etc.

2. The compound of claim 1, wherein X and Y are independently F, Cl, CN, OH, NH₂, NO₂, SO₂NH₂, CO₂NH₂, CH(NOMe), C(O)Me, CO₂Me, CO₂Et, methyl, ethyl, cyclopropyl, CF₃, SMe, OMe, or OEt;
m is 0, 1, 2, 3 or 4; and
n is 0, 1 or 2;
R is —B(OH)₂, —CO₂H, —CONH₂, —C(NH)NH₂, —C(NOH)NH₂, —C(NNH₂)NH₂, —CONHNH₂, —NHNH₂, —NHC(NH)NH₂, —R¹—NHC(O)R¹, —NHSO₂R¹, —NHSO₂R², —NHC(O)NHR², —NHC(S)NHR², —R³;
wherein,
R¹ is (a), (b), (c)-(i) and (iii), (d), (f), (g), (h), (i), (j), (m)-(i), (n), (O), (p), (q), (r), (s), (t), (u), (v); and
wherein, Z is F, Cl, OH, NH₂, NHAC, Me, Et, SMe, OMe, OEt, CHO, CN, CH₂OH, CO₂H, CONH₂, CO₂Me, CO₂Et, or SO₂Me;
q is 0, 1, 2 or 3;
R² is H, methyl, ethyl, cyclopropyl, methylcyclopropyl, cyclopropylmethyl, cyclobutyl, cyclopentyl, cyclohexyl, —CO₂Me, —CO₂Et, 2-oxo-tetrahydro-furan-3-yl, phenyl group substituted up to two times with F, Cl, Br, CN, OH, OMe, SMe, Me, Et, cyclopropyl, CF₃, NMe₂, NO₂, CO₂Et, CO₂Me, SO₂Me, SO₂NH₂ or R³ on the ring;
wherein R³ is azetidin-1-yl, 3-amino-azetidin-1-yl pyrrolidin-1-yl, 3-amino-pyrrolidin-1-yl, 3-amino-4-methyl-pyrrolidin-1-yl, 7-amino-5-aza-spiro[2.4]hept-5-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl or thiomorpholin-4-yl.

3. The compound of claim 2, wherein X and Y are independently F, Cl, CN, OH, NH₂, NO₂, CO₂NH₂, CH(NOMe), methyl, ethyl, cyclopropyl, CF₃, OMe, or OEt;
m is 0, 1, 2, 3 or 4; and
n is 0, 1 or 2.

4. The compound of claim 3, wherein R¹ is (a), (b), (c)-(i) and (iii), (d)-(i) and (iii), (f)-(ii) and (iii), (i), (j)-(ii) and (iii), (m)-(i), (n)-(i), (O)-(ii) and (iii), (p), (q), (r), (s), (t), (u), or (v);
wherein, Z is F, Cl, OH, NH₂, NHAc, Me, Et, SMe, OMe, CHO, CN, CH₂OH, CO₂H, CONH₂, CO₂Me, or CO₂Et;
q is 0, 1, 2 or 3;
R² is H, methyl, ethyl, cyclopropyl, cyclopropylmethyl, cyclobutyl, cyclopentyl, cyclohexyl, —CO₂Me, —CO₂Et, 2-oxo-tetrahydro-furan-3-yl, phenyl group substituted up to two times with F, Cl, Br, CN, OH, OMe, Me, Et, cyclopropyl, CF₃, NO₂, CO₂Et, CO₂Me, SO₂NH₂ or R³ on the ring;
wherein R³ is pyrrolidin-1-yl, 3-amino-pyrrolidin-1-yl, 3-amino-4-methyl-pyrrolidin-1-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl or thiomorpholin-4-yl.

5. A compound selected from the group consisting of:
5-chloro-2-(2,4-dichlorophenoxy)-4-morpholin-4-yl-phenol,
5-chloro-2-(2,4-dichloro-phenoxy)-4-(4-methyl-piperazin-1-yl)-phenol,
5-chloro-2-(2,4-dichloro-phenoxy)-4-thiophen-2-yl-phenol,
5-chloro-2-(2,4-dichloro-phenoxy)-4-furan-2-yl-phenol,
5-chloro-2-(2,4-dichloro-phenoxy)-4-thiophen-3-yl-phenol,
2-chloro-5-(2,4-dichloro-phenoxy)-4-hydroxy-phenylboronic acid,
1-[2-chloro-5-(2,4-dichlorophenoxy)-4-hydroxyphenyl-3-(ethoxycarbonyl)thiourea,
1-[2-chloro-5-(2,4-dichloro-phenoxy)-4-hydroxy-phenyl]-3-(furan-2-carbonyl)-thiourea,
1-[4-hydroxy-3-(2-hydroxy-phenoxy)-phenyl]-3-(ethylozycarbonyl)thiourea,
1-[2-chloro-5-(2,4-dichloro-phenoxy)-4-hydroxy-phenyl]-3-(4-fluoro-phenyl)-thiourea,
1-[2-chloro-5-(2,4-dichloro-phenoxy)-4-hydroxy-phenyl]-3-(4-methoxy-phenyl)-thiourea,
1-[2-chloro-5-(2,4-dichloro-phenoxy)-4-hydroxy-phenyl]-3-cyclohexyl-thiourea,
1-[2-chloro-5-(2,4-dichloro-phenoxy)-4-hydroxy-phenyl]-3-(4-nitro-phenyl)-thiourea,
1-[2-chloro-5-(2,4-dichloro-phenoxy)-4-hydroxy-phenyl]-3-(2-oxo-tetrahydro-furan-3-yl)-thiourea,
1-(3,5-bis-trifluoromethyl-phenyl)-3-[2-chloro-5-(2,4-dichloro-phenoxy)-4-hydroxy-phenyl]-urea,
1-(3,5-bis-trifluoromethyl-phenyl)-3-[2-chloro-5-(2,4-dichloro-phenoxy)-4-hydroxy-phenyl]-thiourea,
5-chloro-2-(2,4-dichloro-phenoxy)-4-pyrrol-1-yl-phenol,
2-(2-hydroxy-5-pyrrol-1-yl-phenoxy)-benzonitrile,
thiophene-2-carboxylic acid [2-chloro-5-(2,4-dichloro-phenoxy)-4-hydroxy-phenyl]-amide,
furan-2-carboxylic acid [2-chloro-5-(2,4-dichloro-phenoxy)-4-hydroxy-phenyl]-amide,
4-methyl-[1,2,3]thiadiazole-5-carboxylic acid [2-chloro-5-(2,4-dichloro-phenoxy)-4-hydroxy-phenyl]-amide,
5-methyl-isoxazole-3-carboxylic acid [2-chloro-5-(2,4-dichloro-phenoxy)-4-hydroxy-phenyl]-amide,
N-{5-[2-chloro-5-(2,4-dichloro-phenoxy)-4-hydroxy-phenylsulfamoyl]-4-methyl-thiazol-2-yl}-acetamide,
1H-imidazole-4-carboxylic acid [2-chloro-5-(2,4-dichloro-phenoxy)-4-hydroxy-phenyl]-amide,
1H-pyrazole-4-carboxylic acid [2-chloro-5-(2,4-dichloro-phenoxy)-4-hydroxy-phenyl]-amide,
2-(2-hydroxy-4-methyl-5-thiophen-2-yl-phenoxy)-benzonitrile,
2-(2-hydroxy-4-methyl-5-thiophen-3-yl-phenoxy)-benzonitrile,
2-(5-furan-2-yl-2-hydroxy-4-methyl-phenoxy)-benzonitrile,
2-(2-hydroxy-4-methyl-5-pyrrol-1-yl-phenoxy)-benzonitrile,
5-chloro-2-(4-fluoro-2-hydroxy-5-morpholin-4-yl-phenoxy)-benzonitrile,
2-(2-hydroxy-4-methyl-5-morpholin-4-yl-phenoxy)-benzonitrile,
5-chloro-2-(2,4-dichloro-phenoxy)-4-tetrazol-1-yl-phenol,
5-chloro-2-(2,4-dichloro-phenoxy)-4-(1H-tetrazol-5-yl)-phenol,
2-chloro-5-(2,4-dichloro-phenoxy)-4,N-dihydroxy-benzamidine,
3-[2-chloro-5-(2,4-dichloro-phenoxy)-4-hydroxy-phenyl][1,2,4]oxadiazole-5-carboxylic acid ethyl ester,
3-[2-chloro-5-(2,4-dichloro-phenoxy)-4-hydroxy-phenyl]-[1,2,4]oxadiazole-5-carboxylic acid,
2-chloro-5-(2,4-dichloro-phenoxy)-4-hydroxy-benzoic acid,
2-chloro-5-(2,4-dichloro-phenoxy)-4-hydroxy-benzamide, and
4-(5-amino-[1,3,4]thiadiazol-2-yl)-5-chloro-2-(2,4-dichloro-phenoxy)-phenol.

6. The compound of claim 1 which is in the form of a prodrug selected from the group consisting of compounds wherein hydroxyl, amine, or sulfhydroxyl groups are bonded to any group that, when administered to an animal, cleave to form a free hydroxyl, amino, or sulfhydroxyl group, respectively.

7. The compound of claim 1 which is in the form of a prodrug selected from the group consisting of acetate, formate, benzoate and phosphate ester derivatives of hydroxyl functional groups, and acetyl and benzoyl derivatives of amine functional groups.

8. The compound of claim 1, wherein the compound comprises tautomeric forms, geometric isomers, enantiomers and diastereomers.

9. The compound of claim 1, wherein the pharmaceutically acceptable salt thereof is an acid addition salt wherein the acid is selected from the group consisting of hydrochloric, sulfuric, phosphoric, acetic, citric, oxalic, malonic, salicyclic, malic, gluconic, fumaric, succinic, ascorbic, maleic, and methanesulfonic acid; or a base salt formed with alkali and alkaline earth metals or organic amines.

10. A composition comprising the following compound of Formula 1 or a pharmaceutically acceptable salt thereof, and a pharmaceutically acceptable carrier, 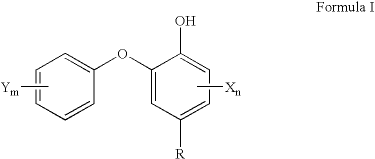
wherein,
X and Y are each halogen, CN, OH, NH₂, NMe₂, NO₂, SO₂Me, SO₃H, SO₂NH₂, CHO, CO₂NH₂, CH(NOMe), C(O)Me, CO₂Me, CO₂Et, C₁-C₄ alkyl, cycloalkyl, CF₃, SMe, OMe, or OEt;
m is 0, 1, 2, 3, 4 or 5; and
n is 0, 1, 2 or 3;
R is —B(OH)₂, —CO₂H, —CONH₂, —C(NH)NH₂, —C(NOH)NH₂, —C(NNH₂)NH₂, —C(O)NHOH, —CONHNH₂, —NHNH₂—NHC(NH)NH₂, —R¹, —NHC(O)R¹, —NHSO₂R¹, —NHSO₂R², —NHC(O)NHR², —NHC(S)NHR², [2-(2-methyl-5-nitro-imidazol-1-yl)-ethyl]oxycarbonylamino or [2-(2-methyl-5-nitro-imidazol-1-yl)-ethyl]oxycarbonyl, or —R³;
wherein R¹ is
(a) Substituted furanyl: 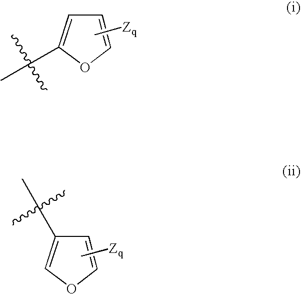
(b) Substituted thiophenyl: 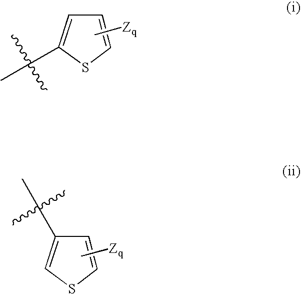
(c) Substituted pyrrolyl: 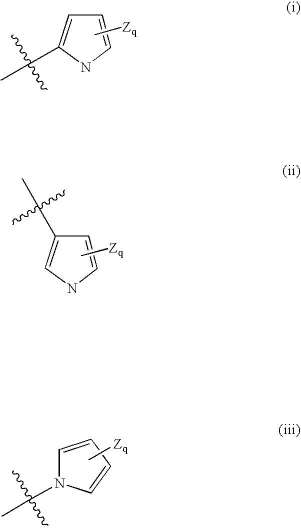
(d) Substituted isoxazolyl 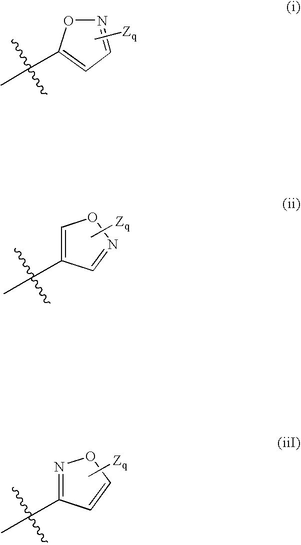
(e) Substituted isothiazolyl 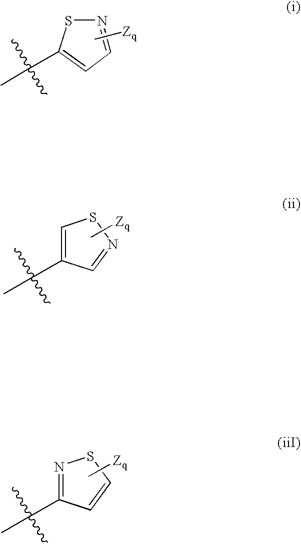
(f) Substituted pyrazolyl 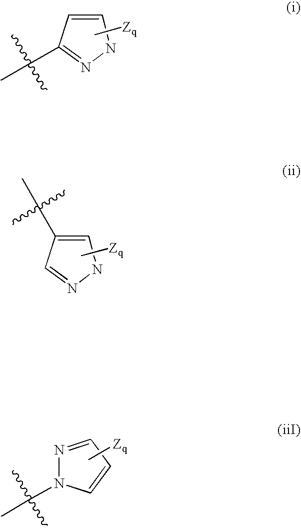
(g) Substituted oxazolyl 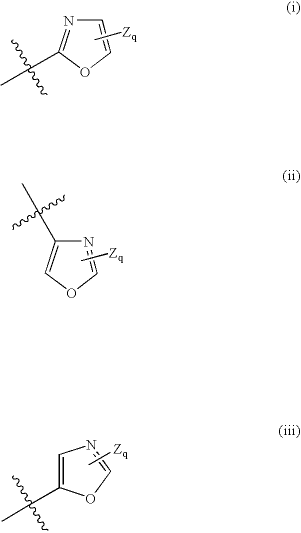
(h) Substituted thiazolyl 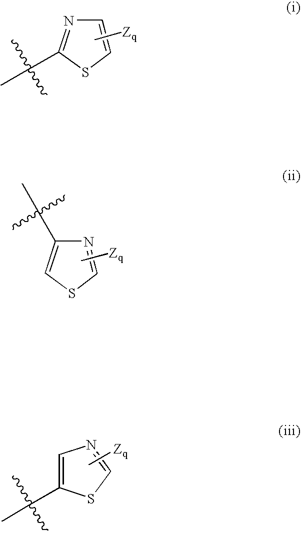
(x) Substituted imidazolyl 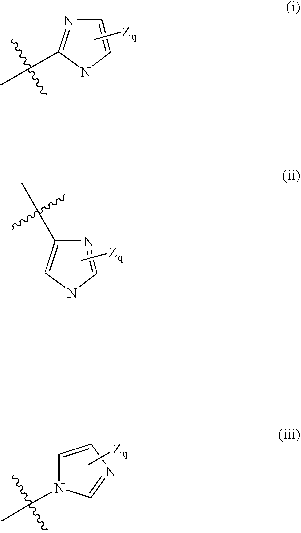
(y) Substituted 1H-[1,2,3]triazolyl 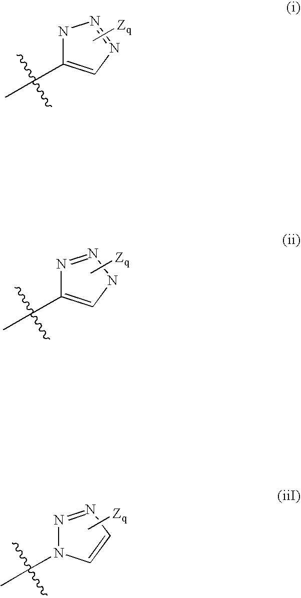
(z) Substituted 2H-[1,2,3]triazol-2-yl 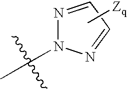
(aa) Substituted [1,2,3]oxadiazolyl 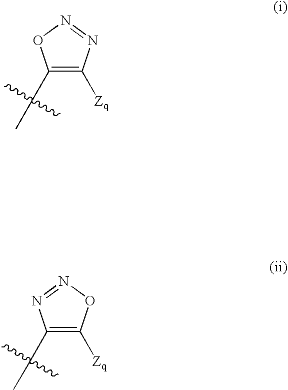
(bb) Substituted [1,2,3]thiadiazolyl 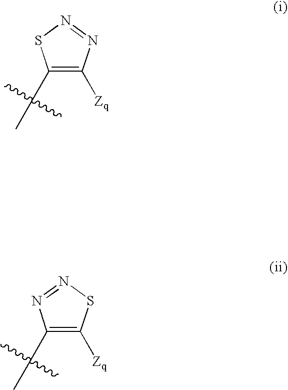
(cc) Substituted 4H-[1,2,4]triazolyl 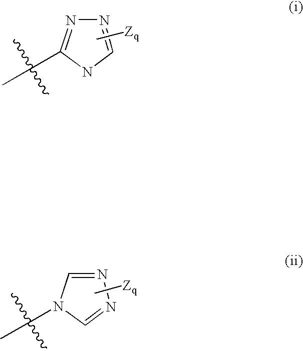
(dd) Substituted 1H-[1,2,4]triazolyl 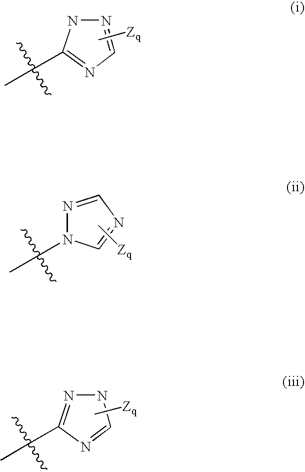
(ee) Substituted [1,3,4]oxadiazolyl 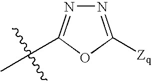
(ff) Substituted [1,3,4]thiadiazolyl 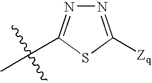
(gg) Substituted [1,2,4]oxadiazolyl 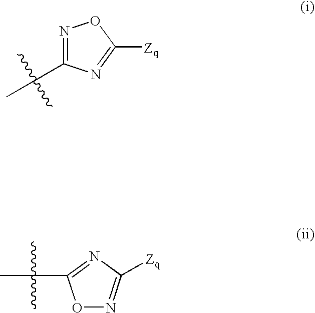
(hh) 1H-Tetrazol-5-yl (i) or 2H-tetrazol-5-yl (ii) 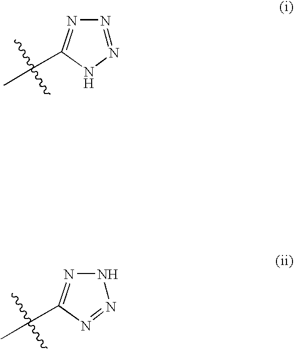
(ii) 1H-Tetrazol-1-yl 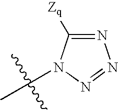
(jj) 5-oxo-4H-[1,2,4]oxadiazol-3-yl 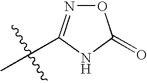
(kk) Substituted 4,5-dihydro-thiazol-2-yl and 5,6-dihydro-4H-[1,3]thiazin-2-yl 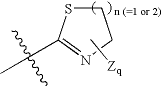
(ll) Substituted pyridazinyl 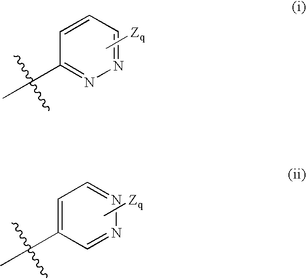
wherein Z is F, Cl, OH, NH₂. NO₂, NMe₂, NHAC, Me, Et, SMe, OMe, OEt, CHO, CN, CH₂OH, CO₂H, CONH₂, CO₂Me, CO₂Et, or SO₂Me;
q is 0, 1, 2 or 3;
R² is H, C₁-C₄ alkyl, C₃-C₇ cycloalkyl, —CO₂Me, —CO₂Et, 2-oxo-tetrahydro-furan-3-yl, 3-pyridinylcarbonyl; phenyl group substituted up to two times with F, Cl, Br, CN, OH, OMe, SMe, Me, Et, cyclopropyl, CF₃, NH₂, NMe₂, NO₂, CO₂Et, CO₂Me, CO₂H, SO₂Me, SO₂NH₂ or R³ on the ring;
wherein R³ is azetidin-1-yl, 3-amino-azetidin-1-yl pyrrolidin-1-yl, 3-amino-pyrrolidin-1-yl, 3-amino-4-methyl-pyrrolidin-1-yl, 7-amino-5-aza-spiro[2.4]hept-5-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl and thiomorpholin-4-yl etc.

11. The composition of claim 10, wherein the carrier is a solid material selected from the group consisting of magnesium carbonate, magnesium stearate, talc, sugar, lactose, pectin, dextrin, starch, gelatin, tragacanth, methyl cellulose, sodium carboxymethyl cellulose, a low melting wax, cocoa butter and mixtures thereof.

12. The composition of claim 10, wherein the carrier is a liquid material selected from the group consisting of water, ethyl alcohol, isopropyl alcohol, ethyl carbonate, ethyl acetate, benzyl alcohol, benzyl benzoate, propylene glycol, 1,3-butylene glycol, oils, glycerol, polyethylene glycols, fatty acid esters of sorbitan, and mixtures thereof.

13. A method of treating or preventing a disease or condition caused by or associated with a microbial infection, which method comprises the administration to an animal in need thereof a pharmaceutical composition comprising an anti-microbial amount of the following compound of Formula 1 or a pharmaceutically acceptable salt thereof, and a pharmaceutically acceptable carrier, 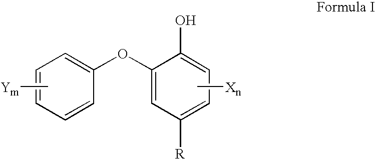
wherein,
X and Y are each halogen, CN, OH, NH₂, NMe₂, NO₂, SO₂Me, SO₃H, SO₂NH₂, CHO, CO₂NH₂, CH(NOMe), C(O)Me, CO₂Me, CO₂Et, C₁-C₄ alkyl, cycloalkyl, CF₃, SMe, OMe, or OEt;
m is 0, 1, 2, 3, 4 or 5; and
n is 0, 1, 2 or 3;
R is —B(OH)₂, —CO₂H, —CONH₂, —C(NH)NH₂, —C(NOH)NH₂, —C(NNH₂)NH₂, —C(O)NHOH, —CONHNH₂, —NHNH₂—NHC(NH)NH₂, —R¹, —NHC(O)R¹, —NHSO₂R¹, —NHSO₂R², —NHC(O)NHR², —NHC(S)NHR², [2-(2-methyl-5-nitro-imidazol-1-yl)-ethyl]oxycarbonylamino or [2-(2-methyl-5-nitro-imidazol-1-yl)-ethyl]oxycarbonyl, or —R³;
wherein R¹ is
(a) Substituted furanyl: 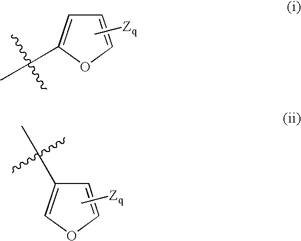
(b) Substituted thiophenyl: 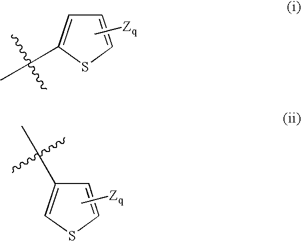
(c) Substituted pyrrolyl: 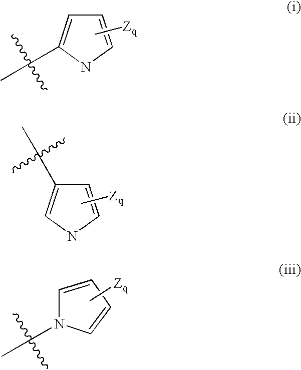
(d) Substituted isoxazolyl 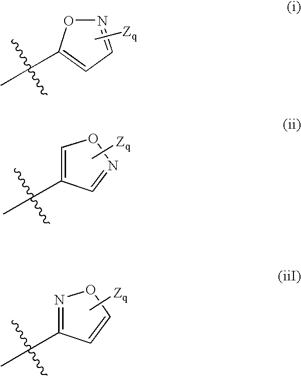
(e) Substituted isothiazolyl 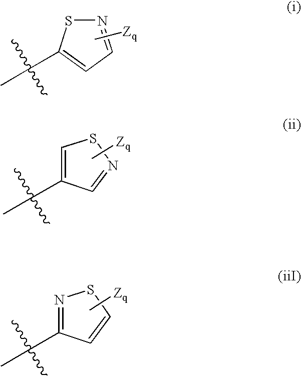
(f) Substituted pyrazolyl 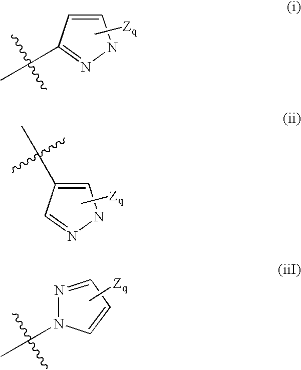
(g) Substituted oxazolyl 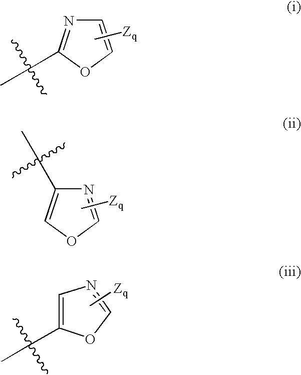
(h) Substituted thiazolyl 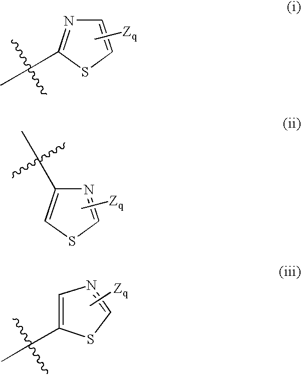
(mm) Substituted imidazolyl 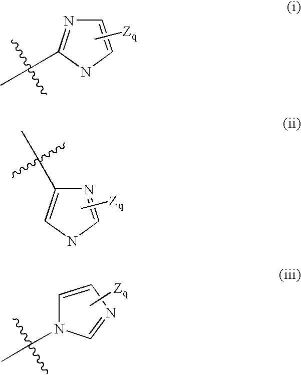
(nn) Substituted 1H-[1,2,3]triazolyl 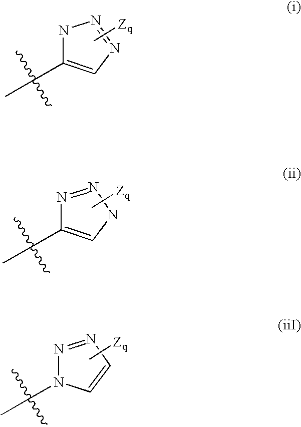
(oo) Substituted 2H-[1,2,3]triazol-2-yl 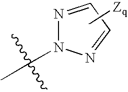
(pp) Substituted [1,2,3]oxadiazolyl 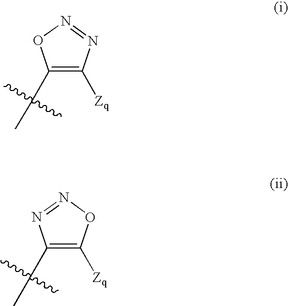
(qq) Substituted [1,2,3]thiadiazolyl 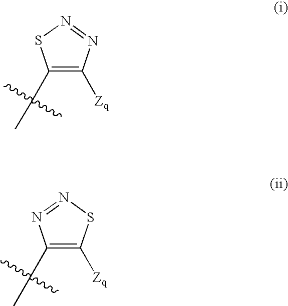
(rr) Substituted 4H-[1,2,4]triazolyl 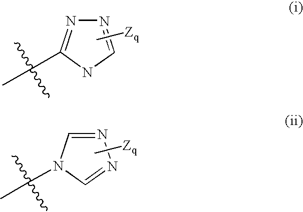
(ss) Substituted 1H-[1,2,4]triazolyl 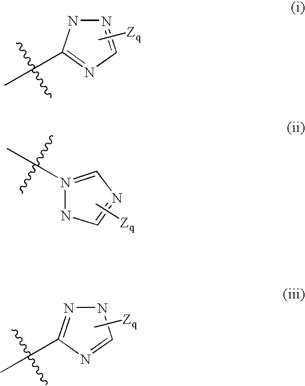
(tt) Substituted [1,3,4]oxadiazolyl 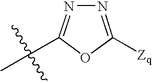
(uu) Substituted [1,3,4]thiadiazolyl 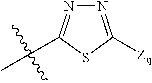
(vv) Substituted [1,2,4]oxadiazolyl 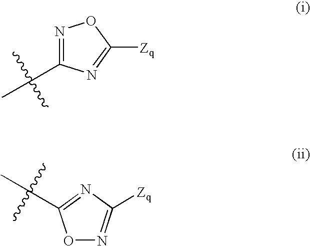
(ww) 1H-Tetrazol-5-yl (i) or 2H-tetrazol-5-yl (ii) 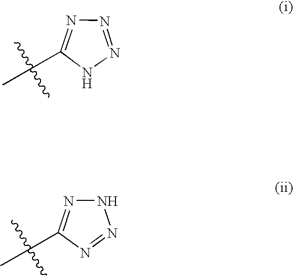
(xx) 1H-Tetrazol-1-yl 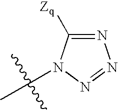
(yy) 5-oxo-4H-[1,2,4]oxadiazol-3-yl 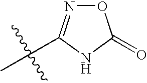
(zz) Substituted 4,5-dihydro-thiazol-2-yl and 5,6-dihydro-4H-[1,3]thiazin-2-yl 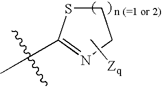
(aaa) Substituted pyridazinyl 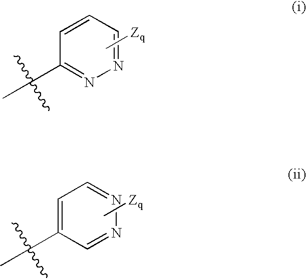
wherein Z is F, Cl, OH, NH₂, NO₂, NMe₂, NHAC, Me, Et, SMe, OMe, OEt, CHO, CN, CH₂OH, CO₂H, CONH₂, CO₂Me, CO₂Et, or SO₂Me;
q is 0, 1, 2 or 3;
R² is H, C₁-C₄ alkyl, C₃-C₇ cycloalkyl, —CO₂Me, —CO₂Et, 2-oxo-tetrahydro-furan-3-yl, 3-pyridinylcarbonyl; phenyl group substituted up to two times with F, Cl, Br, CN, OH, OMe, SMe, Me, Et, cyclopropyl, CF₃, NH₂, NMe₂, NO₂, CO₂Et, CO₂Me, CO₂H, SO₂Me, SO₂NH₂ or R³ on the ring;
wherein R³ is azetidin-1-yl, 3-amino-azetidin-1-yl pyrrolidin-1-yl, 3-amino-pyrrolidin-1-yl, 3-amino-4-methyl-pyrrolidin-1-yl, 7-amino-5-aza-spiro[2.4]hept-5-yl, 3-amino-4-methoxyimino-pyrrolidin-1-yl, piperidin-1-yl, 3-aminopiperidin-1-yl, 4-amino-piperidin-1-yl, piperazin-1-yl, 3-methyl-piperazin-1-yl, 3,5-dimethyl-piperazin-1-yl, 4-methyl-piperazin-1-yl, morpholin-4-yl and thiomorpholin-4-yl etc.

14. The method of claim 13 wherein the composition is administered to at least one of the skin, mouth, eye, respiratory tract, urinary tract, reproductive tract, soft tissues and blood of an animal.

15. The method of claim 13 wherein the animal is a human.

16. The method of claim 13 wherein the composition is applied to the skin of an animal for topical or transdermal administration.

17. The method of claim 16, wherein the composition for topical or transdermal administration is in a form selected from the group consisting of powders, sprays, ointments, pastes, creams, lotions, gels, solutions, patches and inhalants.

18. The method of claim 13 wherein the disease or condition is caused by or associated with infection with a microbe selected from the group consisting of Streptococcus pyogenes, Staphylococcus aureus, methicillin resistant Staphylococcus aureus (“MRSA”), Staphylococcus epidermidis, Bacillus anthracis, Neisseria gonorrhoeae, Neisseria meningitides, Mycobacteria tuberculosis, vancomycin resistant Enterococcae (“VRE”), Helicobacter pylori, Chlamydia pneumoniae, Chlamydia trachomatis, Campylobacter jejuni, Propionibacterium acnes, Pseudomonas aeruginosa, Haemophilus influenzae, Streptococcus pneumoniae, Enterococcus faecalis, Escherichia coli, Corynebacterium diphtheriae, Morazella catarrhalis and Bacillus cereus.

19. The method of claim 13 wherein the composition is administered two or more times.

20. The method of claim 13 wherein the compound is administered in a dose of about 0.0001 to about 100 mg per kilogram of body weight per day

21. The method of claim 13 wherein the compound is administered in an amount of about 0.01 to about 50 mg per kg of body weight per day.

22. The method of claim 13 wherein the compound is administered in a dose of about 0.1 to about 10 mg per kg of body weight per day

23. The method of claim 13 wherein the dose of compound administered is selected from the group consisting of 5, 10, 25, 50, 100, 125, 150, 200, 250 and 500 mg per kg of body weight per day.
